(19) United States
(12) Patent Application Publication
(10) Pub. No.: US 2011/0097401 A1
(43) Pub. Date: Apr. 28, 2011

(54) METHODS FOR TREATING GASTROINTESTINAL DISORDERS

(75) Inventors: Elaine Phillips, San Diego, CA (US); Malcolm Hill, Solana Beach, CA (US); Adam Simpson, San Diego, CA (US)

(73) Assignee: MERITAGE PHARMA, INC., San Diego, CA (US)

(21) Appl. No.: 12/814,335

(22) Filed: Jun. 11, 2010

Related U.S. Application Data
(60) Provisional application No. 61/186,777, filed on Jun. 12, 2009.

(51) Int. Cl. A61K 9/36; A61K 9/28; A61K 9/16; A61K 9/12; A61K 31/60; A61K 31/58; A61P 1/00

(52) U.S. Cl. 424/479; 424/474; 424/497; 424/43; 514/159; 514/174

(57) ABSTRACT

Provided herein are compositions and formulations suitable for the treatment of gastrointestinal disorders. Also provided are methods for treating, preventing, or alleviating disorders of the gastrointestinal tract, for example, those involving the esophagus.

### CROSS-REFERENCE

This application claims the benefit of U.S. Provisional Application No. 61/186,777, filed Jun. 12, 2009, which application is incorporated herein by reference.

### BACKGROUND OF THE INVENTION

There are several disorders of the gastrointestinal tract, e.g., gastrointestinal inflammation, gastrointestinal cancer, gastrointestinal infection, gastrointestinal motility dysfunction, or lesions, wounds or contusions of tissue of a portion of the gastrointestinal tract. In some instances, certain disorders are treated via the systemic administration of a therapeutic agent. In some instances, certain disorders are treated topically, or in a direct manner. In some instances, systemic administration of therapeutic agents leads to undesirable side effects.

### SUMMARY OF THE INVENTION

Provided herein are compositions and formulations suitable for the treatment of gastrointestinal disorders. Also provided are methods for treating, preventing, or alleviating disorders of the gastrointestinal tract, for example, those involving the esophagus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Provided in certain embodiments herein are methods for and compositions useful for preventing or alleviating gastrointestinal inflammation or symptoms thereof in an individual comprising orally administering to said individual a composition comprising at least one therapeutic agent and is formulated to increase the interaction of the composition or therapeutic agent(s) with the gastrointestinal surface (e.g., by comprising at least one excipient that increases the interaction of the composition with a gastrointestinal surface). In some embodiments, the gastrointestinal inflammation is esophageal inflammation. In specific embodiments, the gastrointestinal surface is esophageal epithelium, lamina propria, and/or mucosa. In certain instances, disorders involving the mucosa treated according to a process described herein include inflammation of the epithelium and remodeling of the lamina propria.

In some embodiments, provided herein is a method of preventing or alleviating (i) gastrointestinal inflammation, (ii) gastrointestinal tissue remodeling, and/or (iii) symptoms associated therewith in an individual. In specific embodiments, such a process comprises orally administering to the individual a composition comprising at least one therapeutic agent and at least one excipient that increases the interaction of the composition with a gastrointestinal surface. In some embodiments, the composition is in the form of a powder, granules, micropellets, nanopellets, microparticles, nanoparticles, a tablet, an effervescent tablet, a melting tablet, a disintegrating tablet, an orally disintegrating tablet, a foam, a gel, a solid solution, an emulsion, a liquid or semi-liquid solution, a gum, a wafer (e.g., dissolving or disintegrating), capsule (e.g., dissolving or disintegrating), or a combination thereof. In certain embodiments, the composition is in the form of a film, a patch, a lozenge, or the like.

In further embodiments, provided herein is a method of preventing or alleviating (i) gastrointestinal inflammation, (ii) gastrointestinal tissue remodeling, and/or (iii) symptoms associated therewith in an individual comprising:
a. combining a composition (e.g., solid composition) with a pharmaceutically acceptable liquid to prepare a pharmaceutically administrable composition, the solid composition comprising at least one therapeutic agent and at least one excipient that increases the interaction of the administrable composition with a gastrointestinal surface; and
b. orally administering to said individual the administrable composition.

In some embodiments, the composition is a solid composition is in the form of a powder, granules, micropellets, nanopellets, microparticles, nanoparticles, a tablet, an effervescent tablet, a disintegrating tablet, sachet, or a combination thereof. In certain embodiments, the pharmaceutically acceptable liquid comprises water and/or alcohol. In some embodiments, the solid composition is a powder, granules, micropellets, nanopellets, microparticles, nanoparticles, a tablet, an effervescent tablet, a melting tablet, a disintegrating tablet, a wafer, or the like. In specific embodiments, the solid composition is an effervescent tablet and the liquid comprises water. In other embodiments, the composition to be diluted with a pharmaceutical composition is a non-solid, such as a concentrated solution or suspension, or any other suitable composition described herein (e.g., a gel, emulsion, or the like).

In some embodiments, the compositions described herein are suitable for oral administration and/or are administered orally, in a manner that delivers the composition or an active therapeutic agent contained therein to a gastrointestinal surface. In some embodiments, an orally administered composition is delivered by an orally disintegrating or dissolving formulation, or a powder formulation. In certain embodiments, liquid oral compositions (e.g., suspensions) are utilized in any of the methods described herein. For example, in certain embodiments, a composition described in WO 09/064,457, which is incorporated herein for such disclosure.

In some embodiments, an orally administered composition comprising an orally disintegrating or dissolving composition is swallowed, before, during or after disintegration or dissolution occurs in the oral cavity, in a manner that delivers the composition or an active therapeutic agent contained therein to a gastrointestinal surface. In certain embodiments, delivery of the composition to a gastrointestinal surface (e.g., esophageal surface) is achieved following oral disintegration (at least partial) and/or dissolution (at least partial) in the oral cavity (e.g., in a saliva-composition combination).

In some embodiments, delivery of the composition or active agent, at least in part, occurs during swallowing, resulting in delivery to a portion or entire surface of an upper gastrointestinal surface, e.g., the esophagus. In some embodiments, an oral disintegrating tablet comprising an active therapeutic agent, such as a corticosteroid, is placed in the oral cavity, at least partially dissolves and/or disintegrates, is swallowed, and delivers the active therapeutic agent, such as a corticosteroid, to at least a part of the esophagus.

In some embodiments, an oral formulation comprising an active therapeutic agent, is placed in the oral cavity, is swallowed, and the formulation disperses active ingredient or active ingredient containing particles down the esophagus as the composition is swallowed. In some embodiments, the oral formulation does not substantially disintegrate or dissolve in the oral cavity.

In some embodiments, a tablet, such as an orally disintegrating or dissolving tablet, capsule or any other formulation that has disintegrating and/or dissolving properties, at least in part, and/or that breaks into smaller pieces that the original tablet, capsule or other formulation during administration, is provided to deliver an active therapeutic agent to a gastrointestinal surface.

In some embodiments, a composition described herein is a powder comprising an active therapeutic agent. In some embodiments, a powder composition is administered to the oral cavity, is swallowed, and active ingredient or active ingredient particles are administered to a gastrointestinal surface. In some embodiments, the active ingredient or active ingredient particles are administered to at least a part of an upper gastrointestinal surface, such as the esophagus. In some embodiments, the particles are sprayed or otherwise administered to the oral cavity. In some embodiments a powder or other formulation, such as a suspension, solution, emulsion, etc., comprising an active therapeutic agent is sprayed into the oral cavity. In some embodiments the formulation is sprayed into the back of the mouth or throat. In other embodiments, the powder or other formulation is administered to the mouth, mixed with saliva and swallowed (which may, thereby, deliver a saliva-composition combination to the esophagus).

In some embodiments, an individual suitable for treatment with the compositions disclosed herein may, for example, have been diagnosed with a disorder including, but not limited to, gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. The composition may also be used in treating individuals diagnosed with other gastrointestinal disorders, including stomach and duodenal ulcers, hyperactive acidic discharge disorders, such as Zollinger-Ellison syndrome and laryngeal disorders. In certain embodiments, these methods comprise orally administering to said individual a compositions described herein.

An individual suitable for treatment with the compositions disclosed herein may, for example, have been diagnosed with a disease or condition including, but not limited to, eosinophilic esophagitis, inflammatory bowel diseases involving the esophagus, Crohn's disease, celiac disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, intermediate esophagitis, epithelial hyperplasia, basal cell hyperplasia, elongated papillae, dilated vessels in papillae, fungal esophagitis (e.g., Candida, turolopsis, histoplasma Aspergillus, etc.), viral esophagitis (e.g., HSV, CMV, V2V), bacterial esophagitis (e.g., tuberculosis, actinomycosis, syphilis), corrosive esophagitis, radiation esophagitis, chemotherapy esophagitis, graft vs. host disease, a skin disease with esophageal involvement (e.g., bullous pemphigoid, pemphigus vulgaris, epidermolysis bollosa, Stevens-Johnson syndrome), Behçet's disease, sarcoidosis, idiopathic esophagitis, eosinophilic gastritis, Menetrier's disease, parasitic gastritis, lymphocytic esophagitis, inflammatory bowel disease-associated esophagitis, parasitic gastritis, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation or gastro enteritis. The composition may also be used in treating individuals diagnosed with other gastrointestinal disorders, including stomach and duodenal ulcers, hyperactive acidic discharge disorders, such as Zollinger-Ellison syndrome and laryngeal disorders. In some embodiments, the compositions or methods disclosed herein are used in methods of treating individuals diagnosed with other gastrointestinal disorders, including, by way of non-limiting example, Barrett's Esophagus, gastroesophageal reflux disease (GERD), nonerosive reflux disease (NERD), or erosive esophagitis. In some embodiments, the methods of treating, preventing or alleviating inflammation or symptoms of inflammation include methods of treating any of the gastrointestinal disorders described herein. In certain embodiments, these methods comprise orally administering to said individual a corticosteroid-containing compositions described herein.

Provided herein are methods for treating, preventing and alleviating a disorder that involves the gastrointestinal tract, such as the esophagus, and responds to topical therapy. In some embodiments, the disorder is any disorder described above. The present methods are also useful for treating, preventing or alleviating symptoms and/or inflammation associated with other diseases or conditions of the gastrointestinal tract, for example, the upper gastrointestinal tract, where it is beneficial to target a particular target site, rather than provide systemic therapy. Also provided herein are pharmaceutical compositions useful in the methods of the present application. As used herein, disorders and/or symptoms associated with a disorder disclosed herein includes inflammation and/or symptoms associated with, caused by and/or resulting from the disorder. In some embodiments, provided herein is a method of reducing cytokine and/or chemokine release in the gastrointestinal tract, such as the esophagus (e.g., in the epithelium and/or mucosa thereof) by administering a composition described herein to the gastrointestinal tract (e.g., esophagus). In certain embodiments, provided is a method of decreasing eosinophil, neutrophil and/or mast cell migration to the gastrointestinal tract (e.g., the esophagus) by administering a composition described herein to the gastrointestinal tract (e.g., the esophagus).

In some embodiments, gastrointestinal disorders treated with the methods or compositions described herein include, by way of non-limiting example, gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. In specific embodiments, the gastrointestinal disorder treated with a methods or compositions described herein is an esophageal disorder including, by way of non-limiting example, esophageal inflammation, cancer of the esophagus, esophageal infection (e.g., bacterial or fungal), esophageal motility dysfunction (e.g., achalasia, diffuse esophageal spasm, and nutcracker esophagus), or lesions, wounds or contusions of tissue of an esophageal surface (which is used interchangeably herein with esophageal mucosa and esophageal epithelium). In certain embodiments, gastrointestinal inflammation (e.g., esophageal inflammation) includes, by way of non-limiting example, eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), reflux esophagitis, an inflammatory bowel disease involving the esophagus, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation or gastro enteritis. In specific embodiments, the gastrointestinal inflammation is eosinophilic esophagitis. In some embodiments, the gastrointestinal inflammation is reflux esophagitis, gastroesophageal reflux disease (GERD), nonerosive reflux disease (NERD), Barrett's Esophagus and/or erosive esophagitis. In some embodiments, the gastrointestinal disorder treated with the methods or compositions described herein include, by way of non-limiting example, celiac disease, drug allergy, connective tissue diseases, graft-vs-host disease, oral chronic graft-versus-host disease, radiation injury, chemical injury, oral/esophageal mucositis, oral/esophageal mucositis in cancer patients, and pharyngitis.

As used herein, unless otherwise stated, the use of the terms “a”, “an” and “the” include both singular and multiple embodiments. As used herein, the term “individual” includes any animal. In some embodiments, the animal is a mammal. In certain embodiments, the mammal is a human. In specific embodiments, the human is an adult. In other embodiments, the human is a child (e.g., a child under 12 or a child under 6). In certain embodiments, the human is an infant. As used herein, the phrase “method of treating” or “method for treating” can, in some embodiments, encompass methods of preventing, reducing the incidences of, providing prophylactic treatment, treating and alleviating. As used herein, the phrase “an effective amount” and “a therapeutically effective amount” is an amount sufficient to elicit a change in the symptoms of or a disease state of one or more gastrointestinal disorders, including but not limited to gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. As used herein, the term “or” includes “and” and “or”. Treatment of a disease or disorder herein includes the treatment of the underlying causes of the disease or disorder, symptoms thereof, or the like.

As used herein, the phrase “treating inflammatory diseases involving the esophagus” includes treating symptoms of such diseases and treating inflammation associated with the diseases.

### Actives

In certain embodiments, a composition described herein comprises one or more therapeutic agents. In some embodiments, the at least one therapeutic agent comprises one or more steroids (e.g., a topically active anti-inflammatory steroid).

In some embodiments, the therapeutic agent is a corticosteroid selected from, by way of non-limiting example, aclometasone, amcinonide, beclometasone (beclomethasone), betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone and ulobetasol, and combinations, pharmaceutically acceptable salts and esters thereof. In some embodiments, the corticosteroid is budesonide. In another embodiment, the corticosteroid is an ester of fluticasone, e.g., fluticasone propionate. In some embodiments, the corticosteroid is budesonide, triamcinolone acetonide, triamcinolone alcohol, mometasone, amcinonide, desonide, fluocinonide, fluocinolone acetonide, or halcinonide.

In certain aspects, about 0.1 mg to about 20 mg, about 0.25 mg to about 20 mg, about 0.25 mg to about 15 mg, about 0.25 mg to about 10 mg, or about 0.25 mg to about 5 mg (e.g., about 0.1 to about 5 mg, about 0.3 mg to about 4 mg, about 0.25 to about 2.5 mg, about 0.3 mg to about 2 mg, about 0.5 mg to about 1 mg, about 0.7 mg to about 1.5 mg, about 0.375 mg, about 0.75 mg, about 1 mg, about 1.25 mg, about 1.5 mg or about 2 mg) corticosteroid per day is administered to a patient. In some embodiments, the corticosteroid is present in a unit dose in an amount of between about 0.25 mg and about 5 mg. In some embodiments, the amount of corticosteroid administered daily or in a unit dose is between about 0.1 mg and about 20 mg. In some embodiments, the amount of corticosteroid administered daily or in a unit dose is between about 0.3 mg and about 4 mg. In certain embodiments, the amount of corticosteroid administered daily or in a unit dose is between about 0.5 mg and about 3 mg. In other embodiments, the amount of corticosteroid present in a unit dose or administered daily is between about 1 and about 3 mg, or between about 1 and about 2 mg, or between about 2 and about 3 mg.

In some embodiments, a daily dose of corticosteroid provided according to a method or with a composition described herein is about 0.1 mg/day to about 20 mg/day, about 0.3 mg/day to about 4 mg/day, about 0.3 mg/day to about 5 mg/day. In certain embodiments, a daily dose of corticosteroid (e.g., budesonide) provided according to a method or with a composition described herein is about 0.1 mg/day to about 20 mg/day, about 0.3 mg/day to about 4 mg/day, about 0.3/mg day to about 3 mg/day, about 0.3 mg/day to about 1.5 mg/day, about 0.35 mg/day, about 1.4 mg/day or about 2.8 mg/day for a child aged 2-9. In certain embodiments, a daily dose of corticosteroid (e.g., budesonide) provided according to a method or with a composition described herein is about 0.1 mg/day to about 20 mg/day, about 0.3 mg/day to about 4 mg/day, about 0.3/mg day to about 5 mg/day, about 0.5 mg/day to about 4 mg/day, about 0.5 mg/day to about 2 mg/day, about 0.5 mg/day, about 2 mg/day or about 4 mg/day for a child aged 10-17 and/or an adult. In some embodiments, a composition or dose of a composition described herein comprises about 0.1 mg/day to about 20 mg/day, about 0.3 mg/day to about 4 mg/day, about 0.3 mg to about 2.5 mg, or about 0.35 mg to about 2 mg, or about 0.35 mg, or about 0.5 mg, or about 1.4 mg, or about 2 mg. of corticosteroid (e.g., budesonide).

In some embodiments, the at least one therapeutic agent is selected from the group consisting of a histamine receptor ligand including a histamine antagonist, a transient lower esophageal sphincter relaxation (TLESR)-reducing agent, a promotility agent, a prokinetic serotonergic agent, a potassium-competitive acid blocker (P-CAB), a mucosal protectant, an anti-gastrin agent, an anti-inflammatory agent, an antibacterial agent including an antibiotic, an antifungal agent, a wound healing agent, a chemotherapeutic agent, a monoclonal antibody, an antiemetic agent, erythropoietin, a synthetic erythropoietin, a leukotriene antagonist, a mast cell inhibitor, a mast cell stabilizer, an immunomodulator, an anti-asthmatic agent, a non-steroidal anti-inflammatory drug (NSAID), a corticosteroid, a mGluR₅ antagonist, an acetylcholine modulator, a 5HT₄ receptor agonist, a 5HT₃ receptor antagonist, a 5HT₁ receptor antagonist, an antiseptic agent, an anesthetic, or combinations thereof.

In some embodiments, the at least one therapeutic agent comprises one or more aminosalicyclic acid e.g., 5-aminosalicylic acid (mesalamine) or 4-aminosalicylic acid.

In some embodiments, the therapeutic agent is an H2 receptor ligand (e.g., H2 receptor antagonist). In certain embodiments, H2 receptor antagonists useful herein include, by way of non-limiting example, cimetidine, ranitidine, famotidine and nizatidine. In some embodiments, the therapeutic agent is a H3 receptor ligand (e.g., agonist or antagonist). In certain embodiments, suitable H3 receptor agonists include, by way of non-limiting example, (R)-α-methyl-histamine. In some embodiments, the therapeutic agent is a H1 receptor ligand (e.g., antagonist).

In certain embodiments, the therapeutic agent is a TLESR-reducing agent. Suitable TLESR-reducing agents include, by way of non-limiting example, GABA_{B} agonists (e.g., baclofen), cholecystokinin (CCK-A or CCK-1) antagonists, anticholinergic agents, NO synthase inhibitors, and combinations thereof.

In some embodiments, the therapeutic agent is a prokinetic serotonergic agent. In certain embodiments, suitable prokinetic serotonergic agents include, by way of non-limiting example, 5-HT₄ receptor agonists (e.g., selective 5-HT₄ receptor agonists). 5-HT₄ receptor agonists include, by way of non-limiting example, cisapride, mosapride, tegaserod, and ATI-7505.

In some embodiments, the therapeutic agent is a potassium competitive acid blocker (P-CAB). In certain embodiments, suitable P-CAB include, by way of non-limiting example, soraprazan (BY359), revaprazan (YH1885), AZD0865, CS-526 and combinations thereof.

In certain embodiments, the therapeutic agent is a mucosal protectant. In some embodiments, suitable mucosal protectants include, by way of non-limiting example, sucralfate. In some embodiments, mucosal protectants include one or more of prostaglandin E₂ (PGE₂), epidermal growth factor (EGF) and/or transforming growth factor-α (TGF-α), or analogs thereof. In a specific embodiment, the mucosal protectant comprises the PGE₂ analog trimoprostil.

In certain embodiments, the therapeutic agent is an anti-gastrin agent. In some embodiments, suitable anti-gastrin agents include, by way of non-limiting example, cholecystokinin (CCK-B or CCK-2) antagonists. Cholecystokinin (CCK-B or CCK-2) antagonists include, by way of non-limiting example, Z-360.

In some embodiments, the therapeutic agent is a wound healing agent, an agent that promotes cell survival, an agent that promotes cell proliferation, an antifungal agent, an antibacterial agent, an antibiotic, or a combination thereof.

In further embodiments, the therapeutic agent is a promotility agent. In some embodiments, promotility agents include, by way of non-limiting embodiments, metoclopramide, cisapride, bethanechol, or the like.

In some embodiments, the therapeutic agent is a chemotherapeutic agent. In some embodiments, chemotherapeutic agents include, by way of non-limiting example, 5-fluorouracil, cisplatin, docetaxel, irinotecan, mitomycin, paclitaxel, vindesine, vinorelbine, and the like.

In certain embodiments, the therapeutic agent is a mast cell inhibitor. In some embodiments, suitable mast cell inhibitors include, by way of non-limiting example, cromolyn, cromolyn sodium, nedocromil, and the like. In certain embodiments, the therapeutic agent is a leukotriene antagonist. In some embodiments, suitable leukotriene antagonists include, by way of non-limiting example, montelukast, zafirlukast, zileuton, and the like. In some embodiments, mast cell inhibitors described herein (e.g., montelukast) are present in a composition or dose of a composition described herein in an amount sufficient to provide to an individual about 10 mg/day to about 500 mg/day, about 20 mg/day to about 40 mg/day, about 20 mg/day to about 100 mg/day, or any other therapeutically effective amount.

In some embodiments, the therapeutic agent is a non-steroidal anti-inflammatory drug (NSAID). In specific embodiments, the NSAID is ketoprofen. In various other embodiments, the therapeutic agent is an anti-inflammatory agent, e.g., one as set forth in WO 2008/070129, which reference is hereby incorporated by reference in its entirety.

In some embodiments, the therapeutic agent is an immunomodulator or immunosuppressant. In specific embodiments, the immunomodulator is 6-mercaptopurine (6MP), azathioprine, suplatast tosylate, mycophenolate mofetil, lactobacillus, calcineurin inhibitors (e.g., tacrolimus, cyclosporine, or the like), or the like.

In certain embodiments, the therapeutic agent is a biologic. In specific embodiments, the biologic is an anti IL5, an anti TNF (e.g., TNF-α), an IFN (e.g., IFN-α), an anti-eotaxin-1 antibody, an anti IL-3, an anti IgE, omalizumab, reslizumab, mepolizumab, daclizumab, SCH55700, or the like.

In some embodiments, chemotherapeutic agents include, by way of non-limiting example, imatinib, imatinib mesylate, dasatinib, AMN107, cladribine, or the like.

In some embodiments, the therapeutic agent is an antispasmodic, an agent for treating chest pain (e.g., nitrates, nitroglycerine, or the like), any drug normally administered sublingually (e.g., cardiovascular drugs, vitamins, minerals, or the like), mepoliz, esomepraz, STI571, imatinib, an anti-CD25 (e.g., daclizumab), tyrosine kinase inhibitors, somatostatin, somatostatin analogs, an anti-CCR-3, an anti-TIM-3, ketotifen, a platelet derived growth factor receptor (PDGFR) inhibitor, or the like.

In some embodiments, the at least one therapeutic agent comprises 5-aminosalicylic acid (5-ASA, including free 5-ASA and 5ASA prodrugs), a corticosteroid (including hydrocortisone, prednisone, methylprednisolone, and budesonide), an immunomodulator (including azathioprine [AZA], 6-mercaptopurine [6-MP], cyclosporine, and methotrexate), an anti-TNF agent (including infliximab, adalimumab, and certolizumab), one or more antibiotics (including metronidazole, ciprofloxacin, and rifaximin), or combinations thereof. In some embodiments, the gastrointestinal inflammation is coeliac disease (CD), inflammatory bowel disease (IBD), ulcerative colitis, ulcerative proctitis, left-sided colitis, or universal colitis. In some embodiments, the at least one therapeutic agent comprises 5-aminosalicylic acid (5-ASA, including free 5-ASA and 5ASA prodrugs), a corticosteroid (including hydrocortisone, prednisone, methylprednisolone, and budesonide), an immunomodulator (including azathioprine [AZA], 6-mercaptopurine [6-MP], cyclosporine, and methotrexate), an anti-TNF agent (including infliximab, adalimumab, and certolizumab), one or more antibiotics (including metronidazole, ciprofloxacin, and rifaximin), or combinations thereof, and the gastrointestinal inflammation is eosinophilic esophagitis, coeliac disease (CD), inflammatory bowel disease (IBD), ulcerative colitis, ulcerative proctitis, left-sided colitis, or universal colitis.

In some embodiments, the at least one therapeutic agent comprises 5-ASA (including free 5-ASA and 5ASA prodrugs), one or more antibiotics (including metronidazole, ciprofloxacin, and rifaximin), one or more immunomodulator (including azathioprine [AZA], 6-mercaptopurine [6-MP], cyclosporine, and methotrexate), and one or more anti-TNF agent (including infliximab, adalimumab, and certolizumab).

In some embodiments, the gastrointestinal inflammation is coeliac disease (CD). In some embodiments, the at least one therapeutic agent comprises 5-ASA (including free 5-ASA and 5ASA prodrugs), one or more antibiotics (including metronidazole, ciprofloxacin, and rifaximin), one or more immunomodulator (including azathioprine [AZA], 6-mercaptopurine [6-MP], cyclosporine, and methotrexate), and one or more anti-TNF agent (including infliximab, adalimumab, and certolizumab), and the gastrointestinal inflammation is coeliac disease (CD).

In some embodiments, the disease or condition to be treated by the methods provided is for maintenance of remission of diseases disclosed. In some embodiments, the methods provided are for maintenance of remission of eosinophilic esophagitis, coeliac disease (CD), inflammatory bowel disease (IBD), ulcerative colitis, ulcerative proctitis, left-sided colitis, or universal colitis. In some embodiments, the at least one therapeutic agent comprises 5-ASA (including free 5-ASA and 5ASA prodrugs), one or more antibiotics (including metronidazole, ciprofloxacin, and rifaximin), one or more immunomodulator (including azathioprine [AZA], 6-mercaptopurine [6-MP], cyclosporine, and methotrexate), and one or more anti-TNF agent (including infliximab, adalimumab, and certolizumab), and the methods provided are for maintenance of remission of eosinophilic esophagitis, coeliac disease (CD), inflammatory bowel disease (IBD), ulcerative colitis, ulcerative proctitis, left-sided colitis, or universal colitis.

In specific embodiments, a composition described herein comprises a therapeutically effective dose. Efficacy of treatment of a therapeutically effective dose can be determined in any suitable manner including, e.g., symptom score assessment, gastrointestinoscopy (e.g., esophagogastroduodenoscopy), gastrointestinal (e.g., esophageal) biopsy, histological evaluation, or a combination thereof. For example, processes of diagnosing eosinophilic esophagitis (EoE) and/or determining efficacy of treatment include any suitable process including, by way of a non-limiting example, processes as set forth in Aceves et al., J. Allergy Clin Immunol, February 2008: abstract 270, or Aveves et al., Am J. Gastroenterol., October 2007, 102(10):2271-9, which are both incorporated by reference herein.

In some embodiments, compositions described herein comprise at least two therapeutic agents. In certain embodiments, the at least two therapeutic agents comprise a corticosteroid and at least one additional active agent. In specific embodiments, the at least one additional active agent is selected from, by way of non-limiting example, an acid inhibitor (e.g., a proton pump inhibitor (PPI) or a H2 antagonist), a transient lower esophageal sphincter relaxation (TLESR)-reducing agent, a serotonergic agent/prokinetics, a potassium-competitive acid blocker (P-CAB), a mucosal protectant, a histamine H3 agonist, an anti-gastrin agent, mGluR₅ antagonists, acetylcholine modulator, 5HT₄ receptor agonist, 5HT₃ receptor antagonist, 5HT₁ receptor antagonist, antibiotics, and combinations thereof.

### Excipients

In some embodiments, a composition provided herein comprises one or more therapeutic agent and one or more of the excipients that extends the time the composition is in contact with a surface of the gastrointestinal tract (e.g., the surface of the esophagus) following administration. Excipients that extend the time of contact between the composition and a surface of the gastrointestinal tract (e.g., the surface of the esophagus) include, by way of a non-limiting example, an excipient that increases the viscosity of the composition, imparts a mucoadhesive character upon the composition and/or enhances absorption of the composition through a surface of the gastrointestinal tract (e.g., the surface of the esophagus). In some embodiments, an additional active agent, in addition to treating, preventing or alleviating the symptoms of and/or inflammation associated with inflammatory diseases of the gastrointestinal tract (e.g., esophagus), also serves to extend the time of contact between the composition and a surface of the gastrointestinal tract (e.g., the surface of the esophagus). For example, in certain embodiments, the additional active agent also increases the viscosity of the composition, imparts a mucoadhesive character upon the composition and/or enhances absorption of the composition through a surface of the gastrointestinal tract (e.g., the surface of the esophagus).

In certain embodiments, a composition described herein comprises a sufficient amount of excipient to allow the composition to interact with a surface of the gastrointestinal tract for a sufficient amount of time so as to deliver an effective amount of the composition to the site of gastrointestinal affliction, e.g., the esophagus. In some embodiments, the effective amount of the composition delivered to the gastrointestinal surface is an amount sufficient to coat, or partially coat, a surface of the gastrointestinal surface, and deliver the composition to the affected areas, including by way of example only, the esophagus, the lower esophagus, the esophageal-stomach juncture, the stomach and/or the duodenum. In certain embodiments, the viscosity of the oral dosage form is such that when administered orally, it is not so thick as to cause difficulty in swallowing, cause gagging, and/or be unpalatable. In certain embodiments, the viscosity of the oral dosage form is a viscosity that is sufficient to provide exposure of the therapeutic agent to the esophagus for a sufficient period of time such that the disorder or symptoms of the disorder involving the gastrointestinal tract, including the esophagus, are reduced following administration of a pharmaceutical composition described herein formulated in or as an oral dosage form. In some instances wherein the pharmaceutical composition is in solid or semi-solid form, the composition described has a viscosity, when mixed with saliva or water (e.g., in certain instances wherein the composition is formulated as an effervescent tablet, oral disintegrating tablet, sachet, powder, dissolvable wafer, or the like), the resulting composition (e.g., as orally administered to a human) has a viscosity sufficient to deliver an effective amount of a composition described herein to the gastrointestinal surface (e.g., an inflamed gastrointestinal surface, such as an inflamed esophagus).

In certain embodiments, the excipient or excipients chosen increase the interaction of the composition with the surface of the gastrointestinal tract (e.g., residence time on the surface) by at least 1.02-fold, by at least 1.05-fold, by at least 1.1-fold, by at least 1.2-fold, by at least 1.25-fold, by at least 1.5-fold, by at least 2-fold, by at least 3-fold, by at least 4-fold or by at least 5-fold. In certain embodiments, the increased interaction of the composition is an at least 1.02 fold, by at least 1.05 fold, by at least 1.1 fold, by at least 1.2 fold, by at least 1.25 fold, by at least 1.5 fold, by at least 2 fold, by at least 3 fold, by at least 4 fold or by at least 5 fold of interaction of the composition with esophagus that occurs following passing of the bolus of the composition being swallowed (e.g., 10, 11, 12, 13, 14, 15, 20, 25, 30, 40, 45, 50, 60, 90, 120, or more seconds following initial swallowing of the composition).

In specific embodiments, following oral administration of a composition described herein to the esophagus (e.g., following initial swallowing or drinking of the composition or saliva-composition combination), at least 0.1%, 0.2%, 0.3%, 0.4%, 0.5%, 1%, 2%, 3%, 4%, 5%, 6%, 7%, 8%, 9%, 10%, 15%, 20%, 30%, 40%, 50%, 60%, 70%, 80%, 90% or 95% by weight of the corticosteroid or composition administered is present within the esophagus (e.g., as measured by gamma scintigraphy) after at least 5 seconds, 10 seconds, 15 seconds, 20 seconds, 25 seconds, 30 seconds, 40 seconds, 45 seconds, 50 seconds, or 1, 1.5, 2, 3, 4, 5, 6, 7, 8, 9, 10, 15, 20, 25, 30, 35, 40, 45, 50, 55 or 60 minutes following application of the composition to the esophagus (e.g., swallowing of the composition or composition-saliva combination). In certain instances, even small differences (e.g., increases) in adherence times (e.g., residence times) between formulations can result in therapeutically significant or clinically significant results or improvements.

In some embodiments, a composition (including a saliva-composition combination) or therapeutic agent described herein adheres to or resides upon the esophagus, or some portion thereof, after oral administration for at least 6 seconds, for at least 12 seconds, for at least 15 seconds, for at least 30 seconds, for at least 60 seconds, for at least 90 seconds, for at least 120 seconds, for at least 3 minutes, for at least 4 minutes, for at least 5 minutes, for at least 15 minutes, or for at least 30 minutes. In certain embodiments, the composition (e.g., saliva-composition combination) is retained on the esophagus after oral administration for about 15 seconds to about 120 seconds, or for about 30 to about 90 seconds. In some embodiments, a portion of the (e.g., saliva-composition combination) or therapeutic agent comprises about 90% or more, about 80% or more, about 70% or more, about 60% or more, about 50% or more, about 40% or more, about 30% or more, about 20% or more, about 10% or more, or about 5% or more. In some embodiments, when an oral pharmaceutical composition described herein is administered to an esophagus, e.g., by oral administration, at least 50%, 40%, 30%, 20%, 15%, 10%, 9%, 8%, 7%, 6%, 5%, 4%, 3%, 2%, or 1% of the oral pharmaceutical composition (e.g., saliva-composition combination) adheres to or resides upon/within the esophagus for at least 5 seconds, 10 seconds, 15 seconds, 30 seconds, 45 seconds, 1 minute, 2 minutes, 3 minutes, 5 minutes, 10 minutes, 15 minutes, 20 minutes, 30 minutes, 45 minutes, 1 hour, or more. In certain embodiments, when an oral pharmaceutical composition described herein is administered to the esophagus, e.g., by oral administration, at least 50%, 40%, 30%, 20%, 15%, 10%, 9%, 8%, 7%, 6%, 5%, 4%, 3%, 2%, or 1% of the therapeutic agent (e.g., corticosteroid) adheres to or resides upon the esophagus for at least 5 seconds, 10 seconds, 15 seconds, 30 seconds, 45 seconds, 1 minute, 2 minutes, 3 minutes, 5 minutes, 10 minutes, 15 minutes, 20 minutes, 30 minutes, 45 minutes, 1 hour, or more. In some embodiments, at least 50%, 40%, 30%, 20%, 15%, 10%, 9%, 8%, 7%, 6%, 5%, 4%, 3%, 2%, or 1% of the therapeutic agent (e.g., corticosteroid) adheres to, resides in/on, or is absorbed by the esophagus at least 5 seconds, 10 seconds, 15 seconds, 30 seconds, 45 seconds, 1 minute, 2 minutes, 3 minutes, 5 minutes, 10 minutes, 15 minutes, 20 minutes, 30 minutes, 45 minutes, 1 hour, or more after application of an oral pharmaceutical composition (e.g., saliva-composition combination) described herein is administered to the esophagus, e.g., by oral administration (e.g., swallowing). In certain embodiments, administration of the oral pharmaceutical composition to the esophagus includes orally administering and/or swallowing at least part of the oral pharmaceutical composition or dose of the oral pharmaceutical composition.

In certain embodiments, a composition (including a saliva-composition combination) described has a viscosity sufficient to deliver an effective amount of the composition to the site of gastrointestinal affliction, e.g., the esophagus. In some embodiments, the effective amount of the composition or saliva-composition combination delivered to the esophagus is an amount sufficient to coat, or partially coat, the esophagus, and deliver the composition to the affected areas, including by way of example only, a portion of the esophagus, the lower esophagus, the esophageal-stomach juncture, the stomach and/or the duodenum. In certain embodiments, the viscosity of the composition or saliva-composition combination is such that when administered orally, it is not so thick as to cause difficulty in swallowing, cause gagging, and/or be unpalatable. In certain embodiments, the viscosity of the composition or saliva-composition combination is a viscosity that is sufficient to provide exposure of the therapeutic agent to the esophagus for a sufficient period of time such that the disorder or symptoms of the disorder involving the gastrointestinal tract, including the esophagus, are reduced following administration of a pharmaceutical composition described herein formulated in or as an oral dosage form. In some instances wherein the pharmaceutical composition is in solid or semi-solid form, the composition described has a viscosity, when mixed with saliva (e.g., in the mouth) or water (e.g., in certain instances wherein the composition is formulated as an effervescent tablet, sachet or home brew), the resulting composition has a viscosity sufficient to deliver an effective amount of a composition described herein to the site of gastrointestinal inflammation, e.g., the esophagus.

In certain embodiments, these increases are measured and compared to the measure of an otherwise similar composition lacking the excipient or excipients that increase the interaction of the composition with the surface of the gastrointestinal tract. In certain instances, increased interaction of the composition is measured as a function of the amount of composition present in a select portion of the gastrointestinal tract, including the esophagus (e.g., as measured after the bolus has passed through the esophagus). In specific instances, the amount of composition present in the esophagus is measured in any suitable manner, e.g., by radiolabeling the composition and measuring the amount of the composition in the esophagus utilizing gamma scintigraphy.

An increase in the interaction of the composition with the surface of the gastrointestinal tract (e.g., the surface of the esophagus) may be measured by measuring the retention time of the material along a length of a surface of the gastrointestinal tract (e.g., the surface of the esophagus), wherein the retention time is increased in the presence of the excipients as compared to its absence. In another embodiment, an increased interaction may be measured by the decrease in physiological manifestations or symptoms of the disease or ailment to be treated, including a decrease in total eosinophil counts in a sample of the surface tissue of the gastrointestinal tract (e.g., esophageal surface tissue). In other embodiments, changes in permeability or other cellular characteristics of a surface of the gastrointestinal tract (e.g., the surface of the esophagus) may also be quantified, wherein a change in permeability may indicate an increase in the interaction of the compositions disclosed herein with the surface of the gastrointestinal tract (e.g., the surface of the esophagus).

In certain embodiments, adherence and/or absorption of a pharmaceutical composition or corticosteroid described herein to a gastrointestinal surface site (e.g., esophagus) may be determined in any suitable manner, e.g., by scintigraphy or by an assay. In some embodiments, such determinations are performed in vivo or in vitro. In certain embodiments, in vivo scintigraphy may include combining a pharmaceutical composition described herein with a detectable radioisotope, administering the labeled composition to a subject and detecting and/or measuring the adherence or residence of the pharmaceutical composition or corticosteroid to the gastrointestinal surface (e.g., esophagus) with a device (e.g., camera) that detects and/or measures radioactivity. In some embodiments, in vivo scintigraphy may include linking a corticosteroid described herein with a detectable radioisotope, formulating the labeled corticosteroid into a composition described herein, administering the composition to a subject and detecting and/or measuring the adherence or residence of the pharmaceutical composition or corticosteroid to the gastrointestinal surface (e.g., esophagus) with a device (e.g., camera) that detects and/or measures radioactivity. In certain embodiments, an in vitro assay for detecting adherence of a pharmaceutical composition or corticosteroid described herein to a gastrointestinal mucosal site (e.g., esophagus) may include applying a composition described herein to a distal portion of a strip of gastrointestinal mucosal tissue (e.g., porcine esophageal tissue) and subjecting the composition to a flow of artificial saliva in the direction of the opposite distal portion of the strip. Determination of adherence or residence of the composition and/or corticosteroid may be determined at a given time by detecting either the amount of composition and/or corticosteroid eluted or the amount of composition and/or corticosteroid remaining on the gastrointestinal surface (e.g., esophagus).

In some embodiments, the at least excipient that increases the interaction of the composition with a gastrointestinal surface comprises a viscosity-increasing excipient. Examples of the viscosity-increasing excipient of this type can be found in US 2007/0111978 published May 17, 2007, which is incorporated by reference herein. In certain embodiments, the composition comprises any suitable amount of any one or more excipient that extends the time the composition is in contact with a surface of the gastrointestinal tract following administration, e.g., about 0.1% to about 30% w/w, about 1% to about 26% w/w.

In certain embodiments, pharmaceutical compositions described herein provide delayed delivery of a therapeutic agent. In various embodiments, delivery of the therapeutic agent is systemic and/or local. For example, in certain embodiments, a pharmaceutical composition described herein comprises therapeutic agent and an agent for increasing the gastrointestinal transit time or decreasing the rate of gastrointestinal flow (e.g., a viscosity enhancing agent and/or mucoadhesive agent) of the composition. In some embodiments, delayed delivery of the therapeutic agent results from the increased transit time or decreased gastrointestinal flow rate of the therapeutic agent through the gastrointestinal tract, thereby delaying delivery of the composition and/or therapeutic agent to a point in the gastrointestinal tract where delivery (e.g., systemic or local) occurs (e.g., the esophagus, stomach, small intestines, and/or large intestines).

In certain instances, increased gastrointestinal transit time or decreasing the rate of gastrointestinal flow refers to the transit time or rate of flow of a composition traveling from the oral cavity (mouth) to the stomach (e.g., esophageal transit time or rate of flow along the esophagus). Similarly, in some instances, increased gastrointestinal transit time or decreasing the rate of gastrointestinal flow refers to the transit time or rate of flow of a composition traveling from the esophagus (i.e., entering the stomach) to the duodenum (e.g., stomach transit time or rate of flow in the stomach). Furthermore, in certain instances, increased gastrointestinal transit time or decreasing the rate of gastrointestinal flow refers to the transit time or rate of flow of a composition traveling from the duodenum (i.e., entering the ileum) to the ascending colon (e.g., ileum or small intestine transit time or rate of flow in the ileum or small intestine). Moreover, in some instances, increased gastrointestinal transit time or decreasing the rate of gastrointestinal flow refers to the transit time or rate of flow of a composition traveling through a combination of portions of the gastrointestinal tract, e.g., the esophagus and the stomach; the stomach and the ileum; the esophagus, the stomach and the ileum; or the like.

In some embodiments, the one or more excipient that increases the interaction of the composition with a gastrointestinal surface is selected from one or more cellulose (including cellulose derivatives), dextrose, one or more maltodextrin, hydroxypropylmethyl-cellulose (HPMC), carboxymethyl-cellulose (CMC) (including, e.g., sodium carboxymethyl-cellulose (NaCMC)), microcrystalline cellulose (MCC), carbomer, hydroxyethyl cellulose (HEC), maltodextrin and combinations thereof. In a specific embodiment, the therapeutic agent is a promotility agent. In a further or additional embodiment, the one or more excipient that increases the interaction of the composition with a gastrointestinal surface comprises or is hydroxypropylmethyl-cellulose (HPMC). In a specific embodiment, the one or more excipient that increases the interaction of the composition with a gastrointestinal surface comprises or is carboxymethyl-cellulose (CMC) (including, e.g., sodium carboxymethyl-cellulose (NaCMC)). In a specific embodiment, the one or more excipient that increases the interaction of the composition with a gastrointestinal surface comprises or is microcrystalline cellulose (MCC). In a specific embodiment, the one or more excipient that increases the interaction of the composition with a gastrointestinal surface comprises or is carbomer (i.e., a high molecular weight cross-linked polyacrylic acid). In a specific embodiment, the one or more excipient that increases the interaction of the composition with a gastrointestinal surface comprises or is a combination of CMC and MCC.

In some embodiments, the one or more excipient that increases the interaction of the composition with a gastrointestinal surface is a viscosity enhancing agents. Viscosity-enhancing excipients that may be used in pharmaceutical compositions described herein include, but are not limited to, acacia (gum arabic), agar, aluminum magnesium silicate, sodium alginate, sodium stearate, bladderwrack, bentonite, carbomer, carrageenan, Carbopol, cellulose, microcrystalline cellulose (MCC), ceratonia, chondrus, dextrose, furcellaran, gelatin, Ghatti gum, guar gum, hectorite, lactose, sucrose, maltodextrin, mannitol, sorbitol, honey, maize starch, corn starch, wheat starch, rice starch, potato starch, gelatin, sterculia gum, xanthum gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), carboxymethyl-cellulose (CMC), e.g., sodium carboxymethyl-cellulose (NaCMC), silicon dioxide, polyvinylpyrrolidone (PVP: povidone), Splenda® (dextrose, maltodextrin and sucralose) or combinations thereof. In one non-limiting example, the viscosity-enhancing excipient is Splenda®. In specific embodiments, the viscosity-enhancing excipient is a combination of MCC and CMC (e.g., Avicel RC-591). In some embodiments, the CMC/MCC combination (e.g., Avicel® RC-591) is present in the composition in an amount of about 1 mg/mL to about 150 mg/mL, 1 mg/mL to about 75 mg/mL, or about 5 mg/mL to about 40 mg/mL. In certain embodiments, the CMC/MCC mixed weight ratio is between about 1/99 and about 99/1, about 20/80 and about 5/95, or about 15/85 and about 10/90. In a specific embodiment, the CMC is NaCMC and the CMC/MCC mixed weight ratio is about 11/89. In a specific embodiment, the viscosity enhancing agent is selected from, by way of non-limiting example, Carbopol 974P, Carbopol Ultrez 10, sodium alginate LF120 and sodium alginate H120L. In some embodiments, the viscosity enhancing agent is selected from, by way of non-limiting example, PVP 10,000, PEG 3350 and HiFibro.

In certain embodiments, suitable viscosity enhancing agents include, by way of non-limiting embodiment, one or more cellulose (including cellulose derivatives), dextrose, one or more maltodextrin, hydroxypropylmethyl-cellulose (HPMC), carboxymethyl-cellulose (CMC) (including, e.g., sodium carboxymethyl-cellulose (NaCMC)), microcrystalline cellulose (MCC), carbomer, hydroxyethyl cellulose (HEC), maltodextrin and combinations thereof. In a further or additional embodiment, the viscosity enhancing agent comprises or is hydroxypropylmethyl-cellulose (HPMC). In a specific embodiment, the viscosity enhancing agent comprises or is carboxymethyl-cellulose (CMC) (including, e.g., sodium carboxymethyl-cellulose (NaCMC)). In a specific embodiment, the viscosity enhancing agent comprises or is microcrystalline cellulose (MCC). In a specific embodiment, the viscosity enhancing agent comprises or is carbomer (i.e., a high molecular weight cross-linked polyacrylic acid). In a specific embodiment, the viscosity enhancing agent comprises or is a combination of CMC and MCC.

In specific embodiments, the one or more excipient that increases the interaction of the composition with a gastrointestinal surface comprises or is a mucoadhesive agent. As used herein, mucoadhesive agents are agents that increase the interaction of a composition with a surface of the gastrointestinal tract (e.g., the mucosa and/or epithelium of the gastrointestinal tract or a specific site of the gastrointestinal tract, such as the esophagus). In some embodiments, suitable mucoadhesive agents include, by way of non-limiting example, maltodextrin.

Mucoadhesive agents to be used herein include, by way of non-limiting example, a soluble polyvinylpyrrolidone polymer (PVP), a cadherin (e.g., e-Cad), a carbopol, a crosslinked poly(acrylic acid) (e.g., Carbopol 974P), a carbomer homopolymer, a carbomer copolymer, a water-swellable, but water-insoluble, fibrous, cross-linked carboxy-functional polymer, a hydrophilic polysaccharide gum, one or more maltodextrin, alginate, a cross-linked aliginate gum gel, thiomers (e.g., thiolated chitosan, thiolated polycarbophil, thiolated alginate, thiolated cellulose derivatives, thiolated carboxymethyl cellulose, thiolated polyacrylic acid, or thiolated polyacrylates), PEGylated polymers (e.g., PEGylated polyacrylic acid or PEGylated polyacrylates), lectin, hydroxypropyl methyl cellulose (HPMC), cellulose derivatives, HPMA copolymers, a water-dispersible polycarboxylated vinyl polymer. In some embodiments, the mucoadhesive agent is a carbopol. In a specific embodiment, the mucoadhesive agent is selected from, by way of non-limiting example, Carbopol 974P, Carbopol Ultrez 10, sodium alginate LF120 and sodium alginate H120L. As used herein, a mucoadhesive agent is an agent that adheres to a gastrointestinal surface (e.g., either or both of a gastrointestinal epithelia or mucosa).

In some embodiments, any composition or formulation described herein comprises greater than about 0.2% w/w, greater than about 0.5% w/w, greater than about 1% w/w, greater than about 2% w/w, greater than about 3% w/w, greater than about 4% w/w, greater than about 5% w/w, greater than about 6% w/w, greater than about 7% w/w, greater than about 8% w/w, greater than about 9% w/w, greater than about 10% w/w, greater than about 11% w/w, greater than about 12% w/w, greater than about 13% w/w, greater than about 14% w/w, greater than about 15% w/w, greater than about 16% w/w, greater than about 17% w/w, greater than about 18% w/w, greater than about 19% w/w, greater than about 20% w/w, greater than about 21% w/w, greater than about 22% w/w, greater than about 23% w/w, greater than about 24% w/w, greater than about 25% w/w, greater than about 26% w/w, greater than about 27% w/w, greater than about 28% w/w, greater than about 29% w/w or greater than about 30% w/w of excipient at extends the time the composition is in contact with a surface of the gastrointestinal tract (e.g., the surface of the esophagus). As used herein greater than includes, e.g., up to about 75% w/w, about 80% w/w, about 90% w/w, about 95% w/w, about 98% w/w, or about 99% w/w. In some embodiments, the one or more excipient that increases the interaction of the composition with a gastrointestinal surface comprises at least one maltodextrin. In In certain embodiments, the maltodextrin has a dextrose equivalents (DE) of greater than 4, greater than 5, greater than 10, greater than 11, greater than 12, greater than 13, greater than 14, greater than 15, about 15, about 4 to about 10, about 4 to about 9, about 4 to about 8, about 11 to about 20, about 12 to about 19, about 13 to about 18, or about 14 to about 16. In some embodiments, a composition described herein comprises a first maltodextrin and a second maltodextrin. In specific embodiments, the first maltodextrin has a DE of about 4 to about 10, about 4 to about 9, or about 4 to about 8 and the second maltodextrin has a DE of about 10 to about 20, about 12 to about 19, or about 13 to about 18. Exemplary maltodextrins include, by way of non-limiting example, Maltrin® M040 (with an average DE of about 5), Maltrin® M050 (with an average DE of about 5), Maltrin® M100 (with an average DE of about 10), Maltrin® M150 (with an average DE of about 14-17, or about 15), Maltrin® M180 (with an average DE of about 18), Maltrin® M440 (with an average DE of about 5), Maltrin® M500 (with an average DE of about 10), Maltrin® M510 (with an average DE of about 10), Maltrin® M550 (with an average DE of about 15), Maltrin® M580 (with an average DE of about 18), Maltrin® M700 (with an average DE of about 10), C*Pharm® maltodextrins (with average DE values of about 7, about 14, 18.5, etc.), or the like.

In certain embodiments, pharmaceutical compositions disclosed herein and used herein comprise one or more additional excipients. In addition to excipients that increase the interaction of the composition and/or therapeutic agent with a gastrointestinal surface (e.g., mucoadhesive agents, viscosity enhancing agents, absorption enhancing agents), excipients useful herein include, by way of non-limiting example, binders, fillers, lubricants, solvents, suspension agents, flavoring agents, coloring agents, sweeteners, preservatives, antioxidants, buffering agents, humectants, chelating agents, surfactants, disintegrating agents, and the like.

In some embodiments, provided herein is a composition wherein the composition or therapeutic agent thereof have improved interaction with the surface of a of a composition independent of whether or not an excipient that increases the interaction of the composition or therapeutic agent is present in the composition. In certain embodiments, (e.g., wherein particles, such as micro- or nano-particles, are utilized) formulation and/or processing of the therapeutic agent(s) may itself provide for an improvement in the exposure of the therapeutic agent to the gastrointestinal surface (e.g., upper GI surface, such as an esophageal surface). In some embodiments, the exposure is improved or maximized without increasing or extending the time of contact with the gastrointestinal surface (e.g., as compared to a similarly formulated composition comprising microparticles having a diameter of about 2-3 microns, or comprising budesonide microparticles as formulated in Pulmicort Respules). In certain embodiments, this is achieved by increasing the surface area of the therapeutic agent utilized (e.g., by reducing the particle size of the therapeutic agent, such as by utilizing a composition comprising nanoparticles).

Provided in certain embodiments herein are compositions or formulations comprising a therapeutic agent, optionally one or more excipient that increases the interaction of the composition with a gastrointestinal surface (e.g., an agent that increases viscosity, mucoadhesive character, adsorption to a gastrointestinal surface, absorption of an active to and/or through a gastrointestinal surface, or combinations thereof), optionally one or more binder, optionally one or more filler, optionally one or more lubricant, optionally one or more solvent, optionally one or more suspension agent, optionally one or more flavoring agent, optionally one or more coloring agent, optionally one or more sweetener, optionally one or more preservative, optionally one or more antioxidant, optionally one or more buffering agent, optionally one or more humectant, optionally one or more chelating agent, optionally one or more disintegrating agent, and optionally one or more surfactant. In some embodiments, the composition described herein is a pharmaceutical composition comprising a therapeutic agent, one or more excipient that increases the interaction of the composition with a gastrointestinal surface (e.g., a gastrointestinal epithelium layer and/or a gastrointestinal mucosal layer), one or more of a flavoring agent and/or a sweetener, and one or more of a vehicle and/or a carrier. In further embodiments, the pharmaceutical composition comprises a therapeutic agent, one or more excipient that increases the interaction of the composition with a gastrointestinal surface, one or more of a flavoring agent and/or a sweetener, one or more of a vehicle and/or a carrier, a buffering agent, a surfactant, an optional chelating agent, an optional antioxidant, an optional preservative, an optional flavoring agent, at least one additional excipient, and, optionally, water.

Preservatives include, by way of non-limiting example, benzalkonium chloride, cetrimide (cetyltrimethylammonium bromide), benzoic acid, benzyl alcohol, methyl-, ethyl-, propyl- and butyl-esters of para-hydroxybenzoic acid, chlorhexidine, chlorobutanol, phenylmercuric acetate, borate and nitrate, potassium sorbate, sodium benzoate, sorbic acid, thiomersal (mercurithiosalicylate), combinations thereof, or the like.

Antioxidants include, by way of non-limiting example, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, edetate (EDTA) (e.g., disodium edetate), Diethylenetriaminepentaacetic acid (DTPA), Triglycollamate (NT), combinations thereof, or the like.

Buffering agents include, by way of non-limiting example, citrate buffers (i.e., citric acid and citrate), phosphate buffers, acetate buffers, carbonate buffers (e.g., calcium carbonate, sodium bicarbonate, or the like), hydroxide (e.g., magnesium hydroxide, sodium hydroxide, or the like), combinations thereof, or the like.

Humectants include, by way of non-limiting example, glycerine, propylene glycol, ethylene glycol, glyceryl triacetate, polyols (e.g., sorbitol, xylitol, maltitol, polydextrose), and the like.

Chelating agents include, by way of non-limiting example, edetate (EDTA) (e.g., disodium edetate), Diethylenetriaminepentaacetic acid (DTPA), Triglycollamate (NT), or the like.

In certain embodiments, sweeteners include, by way of non-limiting example, glycerin, acesulfame potassium (AceK), mono-ammonium glycyrrhizinate (e.g., Magnasweet®), sucrose, lactose, glucose, fructose, arabinose, xylose, ribose, mannose, galactose, dextrose, sorbose, sorbitol, mannitol, maltose, cellobiose, xylitol and the like. In specific embodiments, the sweetener includes glycerin, acesulfame potassium and mono-ammonium glycyrrhizinate. Sweeteners are optionally included in any suitable amount including, by way of non-limiting example, about 0.01% w/w to about 60% w/w, about 0.1% w/w to about 30% w/w, about 0.1% w/w to about 5% w/w, about 5% w/w to about 20% w/w, about 0.5% w/w, about 0.8% w/w, about 1% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w or about 19% w/w. In some embodiments, flavoring agents include, by way of non-limiting example, peppermint, orange, bubble gum, wintergreen, grape and cherry. Any suitable amount of flavoring agent is optionally utilized including, e.g., about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 0.01% to about 5% w/w, about 0.01% w/w to about 10% w/w, or about 0.01% w/w to about 50% w/w. In certain embodiments, a composition described herein has a reduced amount of sugar sweetener (e.g., less than 20% w/w, less than 15% w/w, less than 10% w/w, less than 9% w/w, less than 8% w/w, less than 7% w/w, less than 6% w/w, less than 5% w/w, less than 4% w/w, less than 3% w/w, or less than 2% w/w) and/or a preservative to ensure stability of the composition (e.g., to reduce microbe proliferation). In specific embodiments, glycyrrhizinate such as mono-ammonium glycyrrhizinate (e.g., Magnasweet®) is present in an amount of about 0.01% w/w to about 2.95% w/w. In certain embodiments, coloring agents include yellow agents (e.g., FD&C 5 and/or 6), red agents (e.g., FD&C Red 40, Red No. 3), blue, or the like.

Surfactants include, e.g., anionic, cationic, non-ionic, or zwitterionic surfactants, such as, by way of non-limiting example, polysorbate (e.g., polysorbate 20, polysorbate 60, polysorbate 40, polysorbate 80, polysorbate 81, polysorbate 85, polysorbate 120), bile acids or their salts (e.g., sodium taurocholates, sodium deoxytaurocholates, chenodeoxycholic acid, and ursodeoxycholic acid), nonoxynol or polyoxyethylene glycol fatty acid esters, pluronic or poloxamers such as Pluronic F68, Pluronic L44, Pluronic L101, combinations thereof, or the like. In specific embodiments, the surfactant is polysorbate 80.

Furthermore, in some embodiments, provided herein is a pharmaceutical composition formulated in a dosage form for enteric delivery (e.g., in an enteric coated capsule or tablet). In certain embodiments, pharmaceutical compositions formulated in a dosage form for enteric delivery comprise a therapeutically effective amount of a therapeutic agent and an agent that increases the interaction of the composition and/or therapeutic agent with a gastrointestinal surface. In some embodiments, the pharmaceutical composition is formulated in an enteric delivery system (e.g., an enteric coated and/or delayed release pill, an enteric coated and/or delayed release capsule, an enteric coated and/or delayed release granule, an enteric coated and/or delayed release pellet, an enteric coated and/or delayed release tablet, an enteric and/or delayed release matrix, a non-enteric coated viscous and/or mucoadhesive formulation, or the like). In some embodiments, enteric delivery of a pharmaceutical composition described herein allows the pharmaceutical composition to coat or at least partially coat the intestines (e.g., the small intestines, the large intestines or a combination thereof). In certain embodiments, the increased interaction of the pharmaceutical composition with the intestines, or a portion thereof, allows for increased delivery of a therapeutic agent, locally and/or systemically (e.g., as compared to an otherwise similar formulation lacking the agent that increases the interaction of the pharmaceutical composition or therapeutic agent with the gastrointestinal or intestinal surface).

Excipients are used in any suitable amounts, e.g., as described herein. In certain embodiments, the pharmaceutical composition provided herein is stable. In specific embodiments, the pharmaceutical composition is chemically and/or physically stable.

### Formulations

In some embodiments, a composition described herein and comprising a therapeutic agent and an optional excipient that extends the time the composition and/or therapeutic agent is in contact with a surface of the gastrointestinal tract is in the form of powders, granules, micropellets, nanopellets, microparticles (including nanoparticles), a tablet (e.g., oral dissolving tablet and oral disintegrating tablet), a wafer (e.g., dissolving wafer or disintegrating wafer), a chewing gum, a solid solution, an emulsion, a liquid or semi-liquid solution, a liquid suspension, a foam or combinations thereof.

As provided herein, formulations in a form of a powder include finely divided or subdivided preparations, coarsely comminuted products, or products of intermediate particle size. Also a formulation in a form of a powder can be a physical admixture of two or more powdered pure chemical agents present in definite or differing proportions. In addition, powders can contain certain proportions of liquids dispersed thoroughly and uniformly over the solid components of the mixture, or can be composed entirely of solid materials.

A formulation in a form of a powder can comprise particles which are very coarse, of the dimensions of about 10,000 microns or 10 mm, or particles which are extremely fine, approaching dimensions of about 1 micron or less. Sizes of powders can be typically defined as the following:
Very Coarse (or a No. 8) powder—all particles pass through a No. 8 sieve and not more than 20% through a No. 60 sieve.
Coarse (or a No. 20) powder—all particles pass through a No. 20 sieve and not more than 40% through a No. 60 sieve.
Moderately Coarse (or a No. 40) powder—all particles pass through a No. 40 sieve and not more than 40% through a No. 80 sieve.
Fine (or a No. 60) powder—all particles pass through a No. 60 sieve and not more than 40% through a No. 100 sieve.
Very Fine (or a No. 80) powder—all particles pass through a No. 80 sieve. There is no limit as to greater fineness.

In certain embodiments, the therapeutic agent (e.g., corticosteroid(s)) utilized in the compositions herein are utilized as particles. In specific embodiments, the particles are or comprise microparticles and/or nanoparticles. In some embodiments, the microparticles have a mean diameter of about 0.1 microns to about 50 microns. In specific embodiments, the microparticles have a mean diameter of about 1 micron to about 20 microns. In certain embodiments, at least 95%, at least 98%, or at least 99% of the microparticles have a diameter of less than 10 microns. In some embodiments, at least 95%, at least 98%, or at least 99% of the nanoparticles have a diameter of less than 2 microns.

Powders used for the purpose of formulations provided herein include (a) formulations where an individual can mix a directed amount of powder (typically a teaspoon) with a directed amount of water or other liquid followed by swallowing the mixture; and (b) formulations where the powders can be dissolved in warm water or other liquid before use.

For the purpose of formulations provided herein, granules can be of any size suitable for use with the invention. For example, granules can fall within the range of about 4- to about 20-sieve size, or about 12- to about 20-sieve size, although in other embodiments, the ranges are not so limited. Granules can have irregular shapes or a uniformly sphere shape.

Formulations in the form of powders or granules can be reconstituted with water or other liquid before use. Upon reconstitution, the formulations in the form of powders or granules can be mixed with colorants, flavorants, and/or other desired pharmaceutical ingredients, so the reconstituted solution can have pharmaceutical features of a liquid pharmaceutical.

In some embodiments when the composition is in the form of powders or granules, the at least one therapeutic agent and the at least one excipient are mixed and sprayed onto a powdered or granular pharmaceutical support material. The pharmaceutical support material can be selected from, but is not limited to, the group consisting of microcrystalline cellulose and mixtures of microcrystalline cellulose with lactose. Examples of similar powder or granule formulations of this type can be found in US 2005/0009800 published Jan. 13, 2005, which is incorporated by reference herein.

In some embodiments when the composition is in the form of granules, the granules comprise:
(a) one or more amino acids selected from the group consisting of glycine, alanine, valine, leucine, iso-leucine, phenylalanine, proline, aspartic acid, glutamic acid, lysine, arginine, histidine, serine, threonine, cysteine, asparagine and glutamine, and an intragranular polymer; and,
(b) a hydrophilic extragranular polymer in which said granules are dispersed, said extragranular polymer being more rapidly hydrating than said intragranular polymer.

In some embodiments when the composition is in the form of granules comprising a hydrophilic extragranular polymer, the hydrophilic extragranular polymer comprises one or more of the following: polyethylene oxide, polyvinyl acetate, a galactomannan polysaccharide selected from the group consisting of hydroxypropyl guar, guar gum, locust bean gum, pectin, gum acacia, tragacanth and karaya gum, and a cellulose ether.

In some embodiments, the hydrophilic extragranular polymer comprises one or more of the following: polyethylene oxide, a galactomannan polysaccharide selected from the group consisting of hydroxypropyl guar, guar gum, locust bean gum, pectin, gum acacia, gum tragacanth and karaya gum, and cellulose ethers. Certain formulations in the form of granules of this type can be found in US 2003/0219480 published Nov. 27, 2003, which is incorporated by reference herein.

In some embodiments, when the composition is in the form of micropellets, the composition comprises:
(a) from about 10 to 95% by weight of the at least one therapeutic agent;
(b) from about 5 to about 90% by weight of at least one pharmaceutically acceptable binding agent;
(c) from about 0 to about 10% by weight of at least one pharmaceutically acceptable excipient; and
(d) one or more enzyme-friendly organic solvents in an amount sufficient to form an extrudable mixture.

Certain micropellet formulations of this type can be found in WO 2007/020260 published Feb. 22, 2007, which is incorporated by reference herein.

In some embodiments, when the composition is in the form of microparticles or nanoparticles, the composition comprises:
(a) a plurality of microparticles (including nanoparticles) comprising a rapid absorption composition of the at least one therapeutic agent, glyceryl palmitostearate, and macrogol fatty acid ester,
wherein the at least one therapeutic agent is present in an amount of about 30% by weight of a microparticle (including a nanoparticle),
said glyceryl palmitostearate is present in an amount of about 65% by weight of a microparticle (including a nanoparticle) and
said macrogol fatty acid ester is present in an amount of about 5% by weight of a microparticle (including a nanoparticle),
said microparticles (including nanoparticles) coated with at least one-taste masking coating,
(b) a non-cushioning matrix comprising mannitol, microcrystalline cellulose, and crospovidone, wherein
said mannitol is present in an amount of about 45%,
said microcrystalline cellulose is present in an amount of about 15%, and
said crospovidone is present in an amount of about 2% by weight of said dosage form, and
(c) pharmaceutically acceptable excipients, wherein
said taste-masked coated microparticles (including nanoparticles) are dispersed within said matrix.

Certain oral dosage forms containing microparticles (including nanoparticles) of this type can be found in US 2004/0162333 published Aug. 19, 2004, which is incorporated by reference herein.

In some embodiments, when the composition is in the form of microparticles (including nanoparticles), the composition comprises:
(a) microparticles (including nanoparticles) comprising the at least one therapeutic agent;
(b) a film coated composed of
   (i) a pH-independent water-insoluble polymer accounting for 60% or more but less than 80% of the film and
   (ii) a pH-independent water-soluble substance accounting for more than 20% to 40% or less of the film,

wherein the average particle diameter is 350 am or less.

Certain oral dosage forms containing microparticles (including nanoparticles) of this type can be found in US 2005/0175689 published Aug. 11, 2005, which is incorporated by reference herein.

As provided herein, formulations in the form of a tablet include solid dosage forms of medicinal substances prepared with the aid of suitable pharmaceutical excipients. Tablets not only can be used for oral administration but also can be used for sublingual or buccal administration.

In some embodiments when the composition comprises a tablet, the tablet can be a compressed tablet, a multiple compressed tablet, an oral disintegrating tablet, an oral dissolving tablet, a sugar coated tablet, a chocolate-colored tablet, a film coated tablet, an enteric coated tablet, a fast dissolving tablet, a chewable tablet, a buccal tablet, a sublingual tablet, or combinations thereof.

In some embodiments, provided are formulations which comprise a compressed tablet (C.T.). In some embodiments, the C.T. can be prepared using a single compression, comprising the at least one therapeutic agent and a number of pharmaceutical excipients including one or more of the following (a) at least one diluent or filler, (b) at least one binder or adhesive, (c) at least one disintegrator or disintegrating agent, (d) at least one antiadherent, glidant, lubricant or lubricating agent, and/or (e) at least one miscellaneous adjunct including colorants and flavorants. Those skilled in the art will understand choices of commonly used excipients listed above. After compression, compressed tablets can be further coated with various materials if necessary.

In some embodiments, provided are formulations which comprise a multiple compressed tablet (M.C.T.). In some embodiments, the M.C.T. can be prepared using two or more compressions. A multiple compressed tablets can be a multiple-layered tablet or a so-called tablet-within-a-tablet, where an inner tablet comprises a core and an outer portion comprises a shell. Each layer of material can contain a different medicinal agent separated from the others for reasons of incompatibility, for providing drug release in two or more stages, or simply for the unique appearance of a multiple-layered tablet.

In some embodiments when the composition comprises a tablet, the composition comprises
(a) a scleroglucan matrix;
(b) between 5 and 60% by weight of the at least one therapeutic agent; and
(c) between 5 and 40% by weight of a pharmaceutically acceptable excipient.

Certain formulations comprising a scleroglucan matrix of this type can be found in U.S. Pat. No. 5,215,752 issued Jun. 1, 1993, which is incorporated by reference herein.

In some embodiments when the composition comprises a tablet, the composition further comprises a cyclodextrin or a cyclodextrin derivative. In some embodiments, 70% by mass or more of the components in the tablet is cyclodextrin or the cyclodextrin derivative.

Certain formulations comprising a cyclodextrin or a cyclodextrin derivative of this type can be found in US 2006/0172005 published Aug. 3, 2006, which is incorporated by reference herein.

In some embodiments when the composition comprises an oral disintegrating tablet, the composition further comprises
(a) a methacrylic polymer; and
(b) one or more additional excipients;
   wherein said methacrylic polymer and the one or more additional excipient form a single coating layer over the at least one therapeutic agent; and
   wherein the one or more additional excipient is present as an extragranular ingredient.

In some embodiments, the methacrylic polymer is selected from the group consisting of:

poly(ethyl acrylate, methyl methacrylate, trimethylammonioethyl methacrylic chloride) (1:2:0.1),
poly(ethyl acrylate, methyl methacrylate, trimethylammonioethyl methacrylic chloride) (1:2:0.2),
aqueous dispersions containing about 30% of poly(ethyl acrylate, methyl methacrylate, trimethylammonioethyl methacrylic chloride) (1:2:0.1), or
aqueous dispersions containing about 30% of poly(ethyl acrylate, methyl methacrylate, trimethylammonioethyl methacrylic chloride) (1:2:0.2) and mixtures thereof.

Certain disintegrating tablet formulations of this type can be found in U.S. Pat. No. 6,162,466 issued Dec. 19, 2000, which is incorporated by reference herein.

In some embodiments when the composition comprises an oral disintegrating tablet, the composition further comprises
(a) a disintegrant, and
(b) an iron oxide pigment, wherein the elemental iron of the iron oxide pigment is between about 0.10% (w/w) to about 0.22% (w/w) of the total weight of the tablet.

In some embodiments, the disintegrant is selected from the group consisting of alginic acid, carboxymethylcellulose, cellulose, colloidal silicon dioxide, croscarmellose sodium, starch, pregelatinized starch, sodium starch glycolate, polacrilin potassium, crospovidone, magnesium aluminum silicate, methylcellulose, microcrystalline cellulose, povidone and combinations thereof.

Certain disintegrating tablet formulations of this type can be found in WO 2007/008576 published Jan. 18, 2007, which is incorporated by reference herein.

In some embodiments when the composition comprises an oral disintegrating tablet, the composition comprises
(a) about 10-90 wt % of the at least one therapeutic agent;
(b) about 1-40 wt % of a surface controlling compound which is pharmaceutically acceptable in oral compositions and has a water solubility (20° C.) of about 1/1-1/40 (w/w);
(c) about 2-20 wt % of an erosion controlling compound which is pharmaceutically acceptable in oral compositions and has a water solubility of about 1/1-1/10 (w/w);
(d) an amount in the range of about 0.05-1.0 wt %, of a surface activator which is a disintegrating agent for pharmaceutical compositions at which amount the compound is ineffective as a disintegrating agent;
(e) about 0.1-2.0 wt % of a surfactant which is pharmaceutically acceptable in oral compositions and,
(f) about 1-20 wt % of a binder which is pharmaceutically acceptable in oral compositions; or
(g) about 0.5-5.0 wt % of a die wall lubricant which is pharmaceutically acceptable in oral compositions.

Certain disintegrating tablet formulations of this type can be found in U.S. Pat. No. 4,361,545 issued Nov. 30, 1982, and U.S. Pat. No. 4,539,198 issued Sep. 3, 1985, which are both incorporated by reference herein.

In some embodiments when the composition comprises an oral disintegrating tablet, the composition further comprises
(a) about 15% to about 90% by weight of a pharmaceutically-acceptable, powdered hydrocolloid gum, in some embodiments, obtainable from higher plants, and in some embodiments, having a particle size distribution of between about 75 microns to about 300 microns; and
(b) about 5 to about 75% by weight of another dispersion-enhancing, pharmaceutically-acceptable excipient.

Certain disintegrating tablet formulations of this type can be found in U.S. Pat. No. 5,993,860 issued Nov. 30, 1999, which is incorporated by reference herein.

In some embodiments when the composition comprises an oral disintegrating tablet, the composition further comprises
(a) an effervescent base, wherein said effervescent base comprises
   (i) at least one alkaline earth metal carbonate,
   (ii) an organic edible acid, and
   (iii) an alkali metal salt of citric acid; and optionally, and
(b) a pharmaceutically acceptable auxiliary ingredient.

Certain disintegrating tablet formulations of this type can be found in U.S. Pat. No. 6,242,002 issued Jun. 5, 2001, which is incorporated by reference herein.

In some embodiments when the composition comprises an oral disintegrating tablet, the composition further comprises
(a) an excipient; and
(b) erythritol;
   wherein a total amount of said excipient (a) and said erythritol (b) falls within the range of about 30-99% by weight of a total weight of the composition.

Certain disintegrating tablet formulations of this type can be found in U.S. Pat. No. 6,287,596 issued Sep. 11, 2001, which is incorporated by reference herein.

In some embodiments when the composition comprises an oral disintegrating tablet, the composition further comprises at least 80% of mesna, produced by direct compression or compaction, whereby, for example, a tablet of 245 mg has a breaking strength of 50 N and a disintegration time of <3 minutes.

Certain disintegrating tablet formulations of this type can be found in U.S. Pat. No. 6,322,812 issued Nov. 27, 2001, which is incorporated by reference herein.

In some embodiments when the composition comprises an oral disintegrating tablet, the composition comprises
(a) granules having an average particle diameter of 400 μm or less, which granules comprise a composition coated by an enteric coating layer comprising a first component which is an enteric coating agent and a second component which is a sustained-release agent, said composition having 10 weight % or more of the at least one therapeutic agent; and
(b) a water-soluble sugar alcohol.

Certain disintegrating tablet formulations of this type can be found in U.S. Pat. No. 6,328,994 issued Dec. 11, 2001, which is incorporated by reference herein.

In some embodiments when the composition comprises an oral disintegrating tablet, the composition further comprises
(a) a compacted mixture of methylcellulose, for example, having a viscosity of >1000 centipoise; and
(b) an edible calcium or other salt, for example, selected from the group consisting of dibasic calcium phosphate dihydrate, calcium phosphate anhydrous, and tribasic calcium phosphate; or mixtures thereof.

Certain disintegrating tablet formulations of this type can be found in U.S. Pat. No. 6,350,469 issued Feb. 26, 2002, which is incorporated by reference herein.

In some embodiments when the composition comprises an oral disintegrating tablet, the composition further comprises about 40-90 wt. % liquid and about 10-60 wt. % solids whereby said solids are composed of:
(a) about 10-99 wt. % of a polymer mixture comprising or consisting of;
   (i) about 75-99 wt. % of a polymethacrylate copolymer consisting of about 85-98 wt. % of alkyl(meth)acrylate monomers with C₁-C₄ alkyl residues and about 2-15 wt. % of alkyl(meth)acrylate monomers with a quaternary ammonium group in the alkyl residue, and
   (ii) about 1:25 wt. % of an alkali salt of carboxymethylcellulose having a weight average molecular weight of less than 150,000, and
(b) about 1-90 wt. % of at least one pharmaceutically acceptable ingredient.

Certain disintegrating tablet formulations of this type can be found in U.S. Pat. No. 6,656,507 issued Dec. 2, 2003 and US 2001/0055619 published Dec. 27, 2001, which are both incorporated by reference herein.

In some embodiments when the composition comprises an oral disintegrating tablet, the composition comprises
(a) an intraorally releasing first portion, in the form of a molded triturate tablet comprising the at least one therapeutic agent,
wherein the molded triturate tablet comprises an excipient and disintegrates or dissolves within 10 minutes permitting rapid release of the pharmaceutically active ingredient; and
(b) a second releasing portion located around the first portion as a compressed annular tablet, comprising a therapeutic ingredient capable of oral administration and which is releasable and orally ingestible by the individual after the molded triturate has disintegrated or has dissolved intraorally.

Certain disintegrating tablet formulations of this type can be found in U.S. Pat. No. 6,863,901 issued Mar. 8, 2005, U.S. Pat. No. 7,387,792 issued Jun. 17, 2008, US 2003/0035839 published Feb. 20, 2003, US 2003/0118648 published Jun. 26, 2003, and US 2005/0123609 published Jun. 9, 2005, which are all incorporated by reference herein.

In some embodiments when the composition comprises an oral disintegrating tablet, the composition comprises a core material containing the at least one therapeutic agent in a controlled release form, said controlled release form comprising
(a) an outer coating of a polymer selected from aqueous dispersions of enteric methacrylic acid and methacrylic acid esters anionic copolymers having carboxygroup as the functional group or mixtures thereof; and;
(b) an inner coating of a sustained-release copolymer selected from aqueous dispersions of acrylate and methacrylate pH independent, neutral copolymers having quaternary ammonium group as a functional group or mixtures thereof.

Certain disintegrating tablet formulations of this type can be found in U.S. Pat. No. 6,932,981 issued Aug. 23, 2005, and US 2002/0119195 published Aug. 29, 2002, which are both incorporated by reference herein.

In some embodiments when the composition comprises an oral disintegrating tablet, the composition comprises a plurality of units of the at least one therapeutic agent together with one or more pharmaceutical excipients, where the units of the at least one therapeutic agent is present in a matrix comprising
(a) at least one solid paraffin; and
(b) one or more substances from the group of fatty alcohol, triglyceride and fatty acid ester; and wherein the one or more pharmaceutical excipients facilitate rapid disintegration upon oral administration.

Certain disintegrating tablet formulations of this type can be found in U.S. Pat. No. 7,147,869 issued Dec. 12, 2006, and US 2004/0110661 published Jun. 10, 2004, which are both incorporated by reference herein.

In some embodiments when the composition comprises an oral disintegrating tablet, the composition comprises
(a) granules having an average particle diameter of 300 to 400 μm, which granules comprise:
   (i) a core composition comprising excipient and about 10 weight % or more of an acid-labile physiologically active substance,
   (ii) an enteric coating layer for the core composition comprising a first component that is an enteric coating agent and a second component that is a sustained-release agent, and
   (iii) a coating layer comprising mannitol outside the enteric coating layer; and
(b) a water-soluble sugar alcohol, wherein said water-soluble sugar alcohol is in an amount of about 5 to 97 weight % relative to 100 weight % of the orally disintegrable tablet apart from the granules.

Certain disintegrating tablet formulations of this type can be found in U.S. Pat. No. 7,431,942 issued Oct. 7, 2008, which is incorporated by reference herein.

In some embodiments when the composition comprises an oral disintegrating tablet, the composition further comprises
(a) spray-dried mannitol, of which in some embodiments, has at least 80% with an average particle size over 100 μm,
(b) crospovidone, and
(c) one or more pharmaceutically acceptable excipients, and

in some embodiments, wherein the tablet contains no microcrystalline cellulose.

Certain disintegrating tablet formulations of this type can be found in US 2002/0071864 published Jun. 13, 2002, which is incorporated by reference herein.

In some embodiments when the composition comprises an oral disintegrating tablet, the composition further comprises
(a) a diluent, and
(b) a saccharide, and in some embodiments, the saccharide is selected from the group consisting of xylitol, trehalose, maltose, sorbitol, erythritol, glucose, maltitol, mannitol, sucrose, their hydrates, and combinations thereof, and

in some embodiments, wherein the saccharide has a relatively lower melting point than the diluent.

Certain disintegrating tablet formulations of this type can be found in US 2003/0099701 published May 29, 2003, and US 2005/0100599 published May 12, 2005, which are both incorporated by reference herein.

In some embodiments when the composition comprises an oral disintegrating tablet, the composition further comprises
(a) particles including co-spray dried mannitol and sorbitol;
(b) a disintegrant, in some embodiments, selected from the group consisting of crospovidone, croscarmellose, sodium starch glycolate, and combinations thereof; and
(c) a glidant selected from the group consisting of silica gel, and colloidal silica, precipitated silica, and combinations thereof.

Certain disintegrating tablet formulations of this type can be found in US 2003/0118642 published Jun. 26, 2003, which is incorporated by reference herein.

In some embodiments when the composition comprises an oral disintegrating tablet, the composition comprises
(a) 5-40 weight percent of the at least one therapeutic agent;
(b) 40-90 weight percent of a filler;
(c) 0.5-10 weight percent of a binder;
(d) 0.5 to 10 weight percent of a flavoring agent; and
(e) 1-15 weight percent of a disintegrant.

Certain disintegrating tablet formulations of this type can be found in US 2004/0001885 published Jan. 1, 2004, which is incorporated by reference herein.

In some embodiments when the composition comprises an oral disintegrating tablet, the composition further comprises two or more of sugar alcohols and/or saccharides, for example, selected from the group consisting of erythritol, mannitol, lactitol, lactose, glucose, sucrose, maltitol, xylitol, sorbitol, trehalose, and fructose, and in some embodiments,
wherein the difference between a melting point of one having a highest content of said two or more sugar alcohols and/or saccharides and that of any remaining one of said two or more sugar alcohols and/or saccharides is about 5-6° C. or greater.

Certain disintegrating tablet formulations of this type can be found in US 2004/0014680 published Jan. 22, 2004, which is incorporated by reference herein.

In some embodiments when the composition comprises an oral disintegrating tablet, the composition further comprises (a) a saccharide, (b) a polyanionic polymer, (c) a corrigent, and (d) carboxymethylcellulose.

Certain disintegrating tablet formulations of this type can be found in US 2004/0033258 published Feb. 19, 2004, which is incorporated by reference herein.

In some embodiments when the composition comprises an oral disintegrating tablet, the composition further comprises:
(a) a water-soluble acidic substance;
(b) a water-soluble binding agent being soluble in alcoholic solvent; and
(c) a water-soluble saccharide, and
in some embodiments, wherein the water-soluble acidic substance is coated with a water-soluble coating agent being insoluble in an alcoholic solvent.

Certain disintegrating tablet formulations of this type can be found in US 2004/0109890 published Jun. 10, 2004, which is incorporated by reference herein.

In some embodiments when the composition comprises an oral disintegrating tablet, the composition further comprises:
(a) D-mannitol in the form of crystals or fine particles with primary particle average diameter of at least 30 μm and a specific surface area of 0.4 m²/g or less, and
(b) crospovidone.

Certain disintegrating tablet formulations of this type can be found in US 2004/0122106 published Jun. 24, 2004, which is incorporated by reference herein.

In some embodiments when the composition comprises an oral disintegrating tablet, the composition further comprises:
(a) a first matrix forming agent in the form of maltodextrin having a dextrose equivalent (DE) of between 1 and 20, and
(b) a second matrix forming agent in the form of sorbitol.

Certain disintegrating tablet formulations of this type can be found in US 2004/0228919 published Nov. 18, 2004, which is incorporated by reference herein.

In some embodiments when the composition comprises an oral disintegrating tablet, the composition further comprises at least 50% silicified microcrystalline cellulose.

Certain disintegrating tablet formulations of this type can be found in US 2004/0265375 published Dec. 30, 2004, which is incorporated by reference herein.

In some embodiments when the composition comprises an oral disintegrating tablet, the composition further comprises (a) a methylcellulose; (b) a diluent; and (c) crospovidone.

Certain disintegrating tablet formulations of this type can be found in US 2005/0089560, US 2005/0089563, US 2005/0089564, and US 2005/0089565, all published Apr. 28, 2005, which are all incorporated by reference herein.

In some embodiments when the composition comprises an oral disintegrating tablet, the composition further comprises mannitol, xylitol, an inorganic excipient and a disintegrating agent, and in some embodiments, wherein mannitol and xylitol form complex particles and the inorganic excipient and the disintegrating agent are dispersed in the complex particles.

Certain disintegrating tablet formulations of this type can be found in US 2005/0106240 published May 19, 2005, which is incorporated by reference herein.

In some embodiments when the composition comprises an oral disintegrating tablet, the composition comprises granules prepared by granulating a mixture of:
(a) the at least one therapeutic agent;
(b) an adsorbent, and in some embodiments the adsorbent is selected from the group consisting of calcium silicate, light anhydrous silicic acid, synthetic aluminum silicate, silicon dioxide and magnesium metasilicate aluminate, or combinations thereof;
(c) D-mannitol; and
(d) a disintegrator.

Certain disintegrating tablet formulations of this type can be found in US 2005/0147666 published Jul. 7, 2005, which is incorporated by reference herein.

In some embodiments when the composition comprises an oral disintegrating tablet, the composition comprises a compressed blend of:
(a) rapidly dispersing microgranules, for example, prepared by granulating a sugar alcohol or a saccharide or a mixture thereof having an average particle size less than about 30 microns;
(b) a disintegrant; and
(c) a taste-masked microcapsule, in some embodiments, comprising at least one polymeric binder that improves resilience of the microgranules.

Certain disintegrating tablet formulations of this type can be found in US 2005/0232988 published Oct. 20, 2005, which is incorporated by reference herein.

In some embodiments when the composition comprises an oral disintegrating tablet, the composition further comprises:
(a) a calcium carbonate;
(b) a disintegrant, and in some embodiments the disintegrant is selected from the group consisting of sodium starch glycolate, croscarmellose sodium, crospovidone, and combinations thereof; and
(c) a sugar alcohol, and in some embodiments, the sugar alcohol is selected from the group consisting of sorbitol, mannitol, xylitol, erythritol, maltitol, lactitol, and combinations thereof.

Certain disintegrating tablet formulations of this type can be found in US 2005/0244493 published Nov. 3, 2005, US 2007/0196474 published Aug. 23, 2007, and WO 2008/082808 published Jul. 10, 2008, which are all incorporated by reference herein.

In some embodiments when the composition comprises an oral disintegrating tablet, the composition further comprises:
(a) a titanium dioxide, in some embodiments, exhibiting a surface area of from 3.0 to 10.0 m²/g or a calcium phosphate, for example, exhibiting a surface area of between 1 and 10 m²/g;
(b) a disintegrant, and in some embodiments, the disintegrant is selected from the group consisting of sodium starch glycolate, croscarmellose sodium, crospovidone, and combinations thereof; and
(c) a sugar alcohol, and in some embodiments, the sugar alcohol is selected from the group consisting of sorbitol, mannitol, xylitol, erythritol, maltitol, lactitol, and combinations thereof.

Certain disintegrating tablet formulations of this type can be found in US 2007/0196476 published Aug. 23, 2007, US 2007/0196477 also published Aug. 23, 2007, WO 2008/082809 published Jul. 10, 2008, and WO 2008/082810 also published Jul. 10, 2008, which are all incorporated by reference herein.

In some embodiments when the composition comprises an oral disintegrating tablet, the composition comprises:
(a) a core comprising granules of the least one therapeutic agent and a sugar, in some embodiments, selected from the group consisting of sugar alcohol represented by mannitol, xylitol, sorbitol, erythritol, maltitol and maltose; lactose, sucrose, glucose, oligosaccharide, and combinations thereof; and
(b) a coat comprising a pharmaceutical disintegrating agent, for example, selected from the group consisting of crystalline cellulose, low-substituted hydroxypropyl cellulose, carboxymethyl cellulose, calcium carboxymethyl cellulose, crospovidone; or starch represented by potato starch, wheat starch, corn starch, rice starch, hydroxypropyl starch, sodium carboxymethyl starch, partial-pregelatinized starch, and combinations thereof.

Certain disintegrating tablet formulations of this type can be found in US 2006/0134199 published Jan. 22, 2006, which is incorporated by reference herein.

In some embodiments when the composition comprises an oral disintegrating tablet, the composition comprises:
(a) spray-dried mannitol, for example, in a proportion of at least 59.5%;
(b) the at least one therapeutic agent, for example, in a proportion below or equal to about 10% in a form of powders wherein at least 90% in weight of the powders have particle sizes less than 100 μm;
(c) microcrystalline cellulose, for example, in a proportion from about 10 to 18%, with an average particle size of approximately 50 μm where at least 99% in weight of microcrystalline cellulose has particle sizes below 250 μm;
(d) sodium croscarmellose, for example, in a proportion from about 1 to 4%; and
(e) a lubricant agent, for example, in a proportion from about 0.5 to 2% in weight.

Certain disintegrating tablet formulations of this type can be found in US 2006/0165781 published Jul. 27, 2006, which is incorporated by reference herein.

In some embodiments when the composition comprises an oral disintegrating tablet, the composition further comprises:
(a) saccharides, in some embodiments, comprised of a combination of mannitol and one or more of other saccharide(s) selected from sorbitol, erythritol, maltitol, lactose, sucrose, glucose, fructose, maltose, trehalose, paratinit and paratinose are about 40 to 90 parts by weight;
(b) an inorganic excipient, for example, is about 1 to 30 part(s) by weight; and
(c) a disintegrating agent, for example, is about 5 to 40 parts by weight.

Certain disintegrating tablet formulations of this type can be found in US 2007/0275058 published Nov. 29, 2007, which is incorporated by reference herein.

In some embodiments when the composition comprises an oral disintegrating tablet, the composition further comprises:
(a) a plurality of lipid coated active substrates, and in some embodiments, wherein the lipid is a wax or a fatty acid glycerol ester, and any combinations or mixtures thereof; and
(b) at least 10% by weight a silicified excipient.

Certain disintegrating tablet formulations of this type can be found in US 2007/0292508 published Dec. 20, 2007, which is incorporated by reference herein.

In some embodiments when the composition comprises an oral disintegrating tablet, the composition further comprises:
(a) a poly(amino acid);
(b) a delivery agent; and,
(c) a diluent.

Certain disintegrating tablet formulations of this type can be found in US 2008/0255048 published Nov. 16, 2008, and WO 2007/061829 published May 31, 2007, which are both incorporated by reference herein.

In some embodiments when the composition comprises an oral disintegrating tablet, the composition comprises:
(a) granules having an average particle diameter of 400 μm or less, said granules comprising:
   (i) a core composition comprising excipient and 10 weight % or more of the at least one therapeutic agent,
   (ii) an enteric coating layer for the core composition, and
   (iii) a coating layer comprising a water-soluble sugar alcohol outside the enteric coating layer; and
(b) an additive.

Certain disintegrating tablet formulations of this type can be found in US 2008/0292701 published Nov. 27, 2008, which is incorporated by reference herein.

In some embodiments when the composition comprises an oral disintegrating tablet, the composition further comprises:
(a) a filler having an average particle size of about 150 microns or above, for example, in an amount of about 42 to 72% by weight, and wherein said filler is in directly compressible, granulated, compacted or agglomerated form;
(b) silicon dioxide ranging from about 10 to 30% by weight, and in some embodiments, wherein said silicon dioxide covers the surface of said filler.

Certain disintegrating tablet formulations of this type can be found in US 2008/0317853 published Dec. 25, 2008, and WO 2007/074472 published Jul. 5, 2007, which are both incorporated by reference herein.

In some embodiments when the composition comprises an oral disintegrating tablet, the composition further comprises more than one enteric coated sub-tablet, mixed with one or more tablet excipients; wherein the sub-tablets comprise:
(a) an inner core, and in some embodiments, substantially free of any alkaline stabilizing agent, and comprising one or more inert core excipients;
(b) an inert first coating layer, and in some embodiments, substantially free of any alkaline stabilizing agent, disposed over the inner core;
(c) an alkaline stabilizing layer, and in some embodiments, comprising an alkaline stabilizing agent, disposed over the inner core; and
(d) an acid resistant enteric coating layer, and in some embodiments, disposed over the alkaline stabilizing layer.

Certain disintegrating tablet formulations of this type can be found in WO 2007/078271 published Jul. 12, 2007, which is incorporated by reference herein.

In some embodiments when the composition comprises an oral disintegrating tablet, the composition further comprises calcium silicate and at least one water-soluble excipient, in some embodiments, prepared by co-processing, and in some embodiments, wherein said at least one water-soluble excipient is selected from the group consisting of carbohydrate, a water soluble salt or a polyhydric alcohol or its derivative.

Certain disintegrating tablet formulations of this type can be found in WO 2007/113856 published Oct. 11, 2007, which is incorporated by reference herein.

In some embodiments when the composition comprises an oral disintegrating tablet, the composition comprises
(a) a disintegrant; and
(b) individual units wherein each individual unit comprises:
   (i) a core material comprising the at least one therapeutic agent;
   (ii) a controlled release coating layer; and
   (iii) an over-coating layer comprising a mixture of plasticizer agents.

Certain disintegrating tablet formulations of this type can be found in WO 2009/043926 published Apr. 9, 2009, which is incorporated by reference herein.

Additional oral disintegrating tablet formulations of this type can be found in US 2009/0092672 published Apr. 9, 2009, WO 2008/005534 published Jan. 10, 2008, and WO 2009/062993 published May 22, 2009, which are all incorporated by reference herein.

In some embodiments, provided are sugar-coated tablets (S.C.T.) or chocolate-colored tablets (C.C.T.) where compressed tablets or multiple compressed tablets can be coated with a colored or an uncolored sugar. In some embodiments, the sugar coating is water-soluble and can be quickly dissolved. Because coated tablets may be larger and heavier than the original uncoated tablets, formulations defined by weight for these embodiments herein does not include the weight of the sugar coat.

In some embodiments, provided are film-coated tablets (F.C.T.) where compressed tablets or multiple compressed tablets can be further coated with a thin layer of a water-insoluble or water-soluble polymer capable of forming a film over the table. The film coating can use materials more durable than a sugar coating. For example, the film coating can be a non-gelatin film comprising:
(a) carrageenan; (b) modified starch; (c) sorbitol; (d) maltitol; (e) glycerin; and in some embodiments,
(f) water, in some embodiments, in an amount that is less than or equal to about 25 percent by weight.

Certain non-gelatin film of this type can be found in US 2004/0052839 published Mar. 18, 2004, which is incorporated by reference herein.

In some embodiments, provided are enteric-coated tablets (E.C.T.) where tablets described above can be further coated with a coating that resists dissolution or disruption in the stomach but not in the intestines, thereby allowing for tablet transit through the stomach in favor of tablet disintegration and drug dissolution and absorption from the intestines.

In some embodiments when the composition comprises an enteric coated tablet, the composition comprises:
(a) at least one film-forming agent, and in some embodiments, selected from the group consisting of agar, Car-Bopol™ polymers, carboxymethyl cellulose, carboxymethylethyl cellulose, carrageen, cellulose acetate phthalate, cellulose acetate succinate, cellulose acetate trimellitate, chitin, corn protein extract, ethyl cellulose, gum arabic, hydroxypropyl cellulose, hydroxypropylmethyl acetate succinate, hydroxypropyl methylcellulose acetate succinate, hydroxypropyl methylcellulose phthalate, methacrylic acid-ethyl methacrylate-copolymer, methyl cellulose, pectin, polyvinyl acetate phthalate, poly-vinyl alcohol, shellac, sodium alginate, starch acetate phthalate, styrene/maleic acid copolymer and mixtures of said film-forming agents;
(b) at least one plasticizer, and in some embodiments, in an amount of greater than 1.5% by weight relative to the at least one film-forming agent, which plasticizer is selected from the group consisting of triethyl citrate, cetyl alcohol and mixtures of cetyl alcohol and triethyl citrate; and
(c) optionally at least one anti-sticking agent.

Certain enteric coated compositions of this type can be found in WO 2007/020259 published Feb. 22, 2007, which is incorporated by reference herein.

In some embodiments when the composition comprises a tablet suitable for gastrointestinal delivery, the composition comprises:
(a) about 0.01% by weight to about 10.0% by weight of the at least one therapeutic agent;
(b) about 40% by weight to about 98% by weight of a hydrocolloid gum obtainable from higher plants; and
(c) about 2% by weight to about 50% by weight of a pharmaceutically acceptable excipient.

Certain compositions suitable for gastrointestinal delivery of this type can be found in U.S. Pat. No. 5,811,388 issued Sep. 22, 1998, and WO 1996/040078 published Dec. 19, 1996, which are both incorporated by reference herein.

In some embodiments when the composition comprises a tablet suitable for gastrointestinal delivery, the composition comprises:
the at least one therapeutic agent in a bioadhesive polymeric matrix or bioadhesive coated matrix, in some embodiments, wherein the bioadhesive polymer is a water-insoluble hydrophobic polymer selected from the group consisting of polyanhydrides, poly(meth)acrylate, polyhydroxy acids, polyesters, and copolymers thereof.

Certain compositions suitable for gastrointestinal delivery of this type can be found in US 2006/0045865 published Mar. 2, 2006, which is incorporated by reference herein.

In some embodiments when the composition comprises a tablet suitable for gastrointestinal delivery, the composition comprises:
(a) a core comprising the at least one therapeutic agent and one or more pharmaceutical excipient,
wherein the core rapidly releases the at least one therapeutic agent after the core is contacted with a fluid in an environment of use; and
(b) a time-release non-enteric water soluble or water erodible coating surrounding and in contact with the core,
wherein the coating delays the contact of the core with a fluid in an environment of use for a sufficient period of time to delay the release of the at least one therapeutic agent from the core, and wherein, the dosage form excludes an enteric release coating made from an enteric release polymer.

Certain compositions suitable for gastrointestinal delivery of this type can be found in U.S. Pat. No. 6,749,867 issued Jun. 15, 2004, which is incorporated by reference herein.

In some embodiments when the composition comprises a tablet suitable for gastrointestinal delivery, the composition comprises:
(a) about 5 to 40 wt % of the at least one therapeutic agent;
(b) about 30 to 85 wt % of a hydroswelling polymer matrix and
(c) about 2.5 to 5 wt % of a salt being capable of releasing gaseous carbon dioxide in a gastric environment.

Certain compositions suitable for gastrointestinal delivery of this type can be found in WO 2006/000583 published Jan. 5, 2006, which is incorporated by reference herein.

In some embodiments when the composition comprises a tablet suitable for gastrointestinal delivery, the composition further comprises a hydrophilic cellulose ether or a mixture of two or more hydrophilic cellulose ethers.

Certain compositions suitable for gastrointestinal delivery of this type can be found in US 2006/0013874 published Jan. 19, 2006, and WO 2006/005760 published Jan. 19, 2006, which are both incorporated by reference herein.

In some embodiments, provided are fast dissolving tablets where the tablets can be quickly dissolved in the mouth or during or after swallowing. These fast dissolving tablets can contain more than one disintegrating or other agents to facilitate the dissolving and/or disintegration process. Furthermore, these tablets can be made from microparticles or nanoparticles which can dissolve quickly after the tablet disintegrates.

In some embodiments when the composition comprises a fast dissolving tablet, the composition further comprises:
(a) at least one pharmaceutically acceptable dissolution retardant, in some embodiments, selected from the group consisting of ethylcellulose, hydroxypropylmethylcellulose, polyvinylpyrrolidone, Eudragit® EP O and equivalent polymethacrylate products, hydroxypropylethylcellulose and hydroxypropylcellulose, and
(b) at least one pharmaceutically acceptable excipient, in some embodiments, selected from the group consisting of maltose, maltitol, sorbitol, lactose and mannitol.

Certain compositions suitable for gastrointestinal delivery of this type can be found in US 2003/0181501 published Sep. 25, 2003, which is incorporated by reference herein.

In some embodiments when the composition comprises a fast dissolving tablet, the composition further comprises:
(a) at least one water-insoluble hydrophobic inorganic salt, and in some embodiments,
wherein the water-insoluble hydrophobic inorganic salt absorbs not more than 0.2% by weight water at a relative humidity of 95% at 25° C. in combination with
(b) at least one water-insoluble inorganic salt, and in some embodiments,
wherein the water-insoluble inorganic salt absorbs between 0.3% and 3.0% by weight water at a relative humidity of 95% at 25° C.

Certain compositions suitable for gastrointestinal delivery of this type can be found in WO 2008/079342 and WO 2008/079343, both published Jul. 3, 2008, which are both incorporated by reference herein.

In some embodiments, provided are chewable tablets, in some embodiments, such chewable tablets have a smooth, rapid disintegration when chewed or allowed to dissolve in the mouth, often resulting a creamy base in the mouth. Chewable tablets can be useful in tablet formulations for children and can be prepared by compression.

In some embodiments when the composition comprises a chewable tablet, the composition comprises:
(a) a therapeutically-effective amount of the at least one therapeutic agent dispersed in a solid pharmaceutically-acceptable lipid coating, in some embodiments, which lipid is solid at ambient temperature, or mixtures of said lipids, and in some embodiments, wherein the lipid is present in the composition in an amount of from 5-50 percent by weight.
(b) a matrix for said drug and lipid, said matrix consisting essentially of:
   (i) one or more granulating agents,
   (ii) a rapid dispersal agent, and in some embodiments, in an amount of from about 2 to about 20 weight percent of the composition, wherein the rapid dispersal agent is blended with the solidified lipid coated drug, and
   (iii) optionally minor amounts of additives selected from the group consisting of flavoring agents, coloring agents, buffering agents, sweeteners, oils, and surfactants.

Certain chewable tablet formulations of this type can be found in U.S. Pat. No. 5,320,848 issued Jun. 14, 1994, which is incorporated by reference herein.

In some embodiments when the composition comprises a chewable tablet or an oral disintegrating tablet, the composition comprises:
(a) about 50% of the at least one therapeutic agent;
(b) about 10-12% of a mixture, in some embodiments, containing 87 parts by weight of microcrystalline cellulose and, in some embodiments, 13 parts by weight of guar gum;
(c) about 9.5-29% of a cross-linked polyvinylpyrrolidone;
(d) about 2.5-10% of colloidal silicon dioxide;
(e) about 4-19% of dibasic calcium phosphate; and
(f) about 9-21% of mannitol or maltitol.

Certain chewable tablet or oral disintegrating tablet formulations of this type can be found in U.S. Pat. No. 6,080,427 issued Jun. 27, 2000, which is incorporated by reference herein.

In some embodiments when the composition comprises a chewable tablet or an oral disintegrating tablet, the composition comprises:
(a) a plurality of particles comprising the at least one therapeutic agent, said particles having a particle size of about 150 μm to about 400 μm; and
(b) a matrix comprising from about 0.1 percent to about 25 percent of hydroxyalkylcellulose having a weight average molecular weight of from about 60,000 to about 5,000,000.

Certain chewable tablet or oral disintegrating tablet formulations of this type can be found in US 2004/0265372 published Dec. 30, 2004, US 2004/0265373 published Dec. 30, 2004, and US 2009/0104267 published Apr. 23, 2009, which are all incorporated by reference herein.

In some embodiments when the composition comprises a chewable tablet or an oral disintegrating tablet, the composition further comprises from about 0.25 percent to about 5 percent of polyethylene oxide having an average molecular weight of from about 500,000 to about 10,000,000.

Certain chewable tablet or oral disintegrating tablet formulations of this type can be found in U.S. Pat. No. 6,753,009 issued Jun. 22, 2004, US 2003/0175336 published Sep. 18, 2003, and US 2003/0175339 published Sep. 18, 2003, which are all incorporated by reference herein.

In some embodiments when the composition comprises a chewable tablet or an oral disintegrating tablet, the composition further comprises a matrix comprising directly compressible dextrose monohydrate and sucralose, said tablet containing less than 5% fat and said matrix being substantially free of non-saccharide, water soluble polymeric binders.

Certain chewable tablet or oral disintegrating tablet formulations of this type can be found in US 2002/0122823 published Sep. 5, 2002, which is incorporated by reference herein.

In some embodiments when the composition comprises a chewable tablet or an oral disintegrating tablet, the composition further comprises enteric coated particles. Certain chewable tablet or oral disintegrating tablet formulations comprising enteric coated particles of this type can be found in US 2006/0078611 and US 2006/0078612, both published Apr. 13, 2006, which are both incorporated by reference herein.

In some embodiments, provided are buccal or sublingual tablets where flat-shaped tablets are used in the buccal pouch (for buccal tablets) or beneath the tongue (for sublingual tablets). Buccal and sublingual tablets can be prepared to erode or to dissolve slowly to provide an extended effect.

In some embodiments of a tablet formulation, the composition further comprises
(a) a bioadhesive material, said bioadhesive material providing for adherence to the oral mucosa of said subject; and
(b) stearic acid.

Certain formulations of this type can be found in US 2008/0116404 published Jul. 10, 2008, which is incorporated by reference herein.

In some embodiments of a tablet formulation, the composition comprises
(a) a compressed core devoid of pharmaceutically active substance and comprising at least one diluting agent, and
(b) a coating comprising the at least one therapeutic agent.

Certain formulations of this type can be found in US 2008/0226717 published Sep. 18, 2008, which is incorporated by reference herein.

In some embodiments of a tablet formulation, the composition comprises
(a) about 48.5% of the at least one therapeutic agent;
(b) about 44.5% microcrystalline cellulose;
(c) about 5% low-substituted hydroxypropyl cellulose; and
(d) about 2% magnesium stearate.

Certain formulations of this type can be found in WO 2007/028247 published Mar. 15, 2007 published Aug. 21, 2008, which is incorporated by reference herein.

In some embodiments of a tablet formulation, the composition comprises a non-mucoadhesive orally disintegrating film comprising
(a) a hydrophilic binder and
(b) a water-soluble diluent.

Certain formulations of this type can be found in WO 2008/040534 published Apr. 10, 2008, which is incorporated by reference herein.

In some embodiments of a tablet formulation, the composition comprises
(a) about 0.5% to about 90% of the at least one therapeutic agent;
(b) about 7.5% to about 95% filler; and
(c) about 2.5% to about 10.5% disintegrant.

Certain formulations of this type can be found in WO 2008/095284 published Aug. 14, 2008, which is incorporated by reference herein.

In some embodiments of a tablet formulation, the composition further comprises
(a) methylsulfonylmethane; and
(b) a carrier comprises at least one of an edible oil; water and lecithin.

Certain formulations of this type can be found in WO 2008/099397 published Aug. 21, 2008, which is incorporated by reference herein.

In some embodiments when the composition comprises powders, granules, and/or a tablet, the composition further comprises:
(a) a solid carrier, in some embodiments, selected from magnesium aluminometasilicate, dibasic calcium phosphate, or both, which carrier contains the at least one therapeutic agent or a liquid having one or more components selected from the group consisting of the at least one therapeutic agent, oral absorption enhancers, and solubility enhancers, and
(b) pharmaceutical processing aids, in some embodiments, selected from one or more glidants, disintegrants, lubricants, and/or bulking agents.

Certain solid dosage forms having a solid carrier containing a liquid component of this type can be found in U.S. Pat. No. 6,793,934 issued Sep. 21, 2004, which is incorporated by reference herein.

In some embodiments when the composition is in the form of a solid solution, the composition comprises
(a) up to about 65% by weight of the at least one therapeutic agent;
(b) at least about 10% by weight of an alkaline compound or a mixture of alkaline compounds; and
(c) from about 0.1 to about 10% by weight of one or more surfactants.

In some embodiments when the composition is in the form of a solid solution, the composition comprises
(a) up to about 50% by weight of the at least one therapeutic agent;
(b) from about 20 to about 70% by weight of a non-ionic hydrophilic surfactant which is liquid between about 15 and about 30° C.; and
(c) from about 5 to about 70% by weight of a pharmaceutically acceptable organic polymer or polymer mixture, in some embodiments, which is liquid above about 60° C. and solid below about 30° C.

In some embodiments, the non-ionic hydrophilic surfactant is selected from the group consisting of polyoxyethylene glycol sorbitan fatty acid esters, non hydrogenated polyoxyethylene castor oil derivatives, and combinations thereof.

In some embodiments, the pharmaceutically acceptable organic polymer is a polyethylene glycol or a mixture of polyethylene glycols, each with a molecular weight of between 1000 and 50000 Daltons.

In some embodiments, the solid solution further comprises a disintegrating agent in an amount of between about 1% and about 10% by weight.

Certain solid solution formulations of this type can be found in US 2005/0008697 published Jan. 13, 2005, WO 2003/068266 published Aug. 21, 2003; and WO 2006/067150 published Jun. 29, 2006, which are all incorporated by reference herein.

In some embodiments when the composition is in the form of an emulsion, the emulsion has a hydrophilic phase and a lipophilic phase, and the composition comprises
(a) an enzyme or enzyme mixture having at least lipolytic activity, and
(b) a system comprising at least one surfactant, and at least one co-surfactant.

In some embodiments, the system for the emulsion above comprises:
(a) a surfactant, in some embodiments, selected from the group consisting of polyethylene glycol fatty acid mono- and/or di-esters with aliphatic C₆-C₂₂ carboxylic acids; polyethylene glycol glycerol fatty acid esters with aliphatic C₆-C₂₂ carboxylic acids; polyethylene glycol alkyl mono- and/or di-ethers with aliphatic C₁₂-C₁₈ alcohols, and mixtures thereof;
(b) a co-surfactant, in some embodiments, selected from the group consisting of mono-acylglycerides with aliphatic C₆-C₂₂ carboxylic acids, mono-ethers of glycerol ethers with aliphatic C₁₂-C₁₈ alcohols, partial esters of propyleneglycol with aliphatic C₆-C₂₂ carboxylic acids, partial esters of polyglycerol with aliphatic C₆-C₂₂ carboxylic acids, and mixtures thereof, and
(c) a lipophilic phase, in some embodiments, represented by di- and/or triacylglycerides with aliphatic C₆-C₂₂ carboxylic acids.

In some embodiments, the system for the emulsion above comprises:
(a) a surfactant selected from the group consisting of polyethylene glycol fatty acid esters; polyethylene glycol glycerol fatty acid esters; polyethylene glycol alkyl ethers, polyethylene glycol sterol ethers, polyethylene glycol sorbitan fatty acid esters, sugar esters, polyoxyethylene-polyoxypropylene block copolymers, ionic surfactants and mixtures thereof;
(b) a co-surfactant selected from the group consisting of mono-acylglycerides, mono-ethers of glycerol, partial esters of propyleneglycol, partial esters of polyglycerol, partial esters of ethyl diglycol and mixtures thereof, and
(c) a lipophilic phase represented by di- and/or triacylglycerides.

Certain emulsion formulations of this type can be found in US 2005/0250817 published Nov. 10, 2005 and WO 2005/092370 published Oct. 6, 2005, which are both incorporated by reference herein.

In some embodiments when the composition comprises an emulsion, the composition further comprises
(a) an oily medium, and in some embodiments, selected from the group consisting of triglycerides, mixed glycerides, free fatty acids having from C₆ to C₃₂ carbon atoms, and mixtures thereof; and
(b) a surfactant, in some embodiments, which promotes self-emulsification.

Certain emulsion formulations of this type can be found in WO 2007/056242 published May 18, 2007, which is incorporated by reference herein.

In some embodiments when the composition comprises an emulsion, the composition comprises
(a) an admixture of an emulsion comprising the at least one therapeutic agent; and
(b) a solid particle adsorbent; and in some embodiments, wherein said emulsion has a viscosity of between about 400 cps and about 200,000 cps, emulsion globules having diameters of between about 120 nm and about 70 μm, and wherein said emulsion is adsorbed on said solid particle adsorbent.

Certain emulsion formulations of this type can be found in U.S. Pat. No. 6,692,771 issued Feb. 17, 2004, US 2002/0160049 published Oct. 31, 2002, and US 2003/0077306 published Apr. 24, 2003, which are all incorporated by reference herein.

In some embodiments when the composition is in the form of a liquid or semi-liquid solution, the composition comprises:
(a) up to about 50% by weight of the at least one therapeutic agent;
(b) a non-ionic hydrophilic surfactant ingredient, and in some embodiments, which is in the liquid form between about 15° C. and about 30° C., selected from the group consisting of polyoxyethylene glycol sorbitan fatty acid esters (polysorbates) and non hydrogenated polyoxyethylene castor oil derivatives, said non-ionic hydrophilic surfactant having a hydrophilic-lipophilic balance (HLB) value of between 14 and 16.

Certain liquid or semi-liquid solution formulations of this type can be found in WO 2004/062692 published Jul. 29, 2004, which is incorporated by reference herein.

In some embodiments when the composition comprises a liquid dosage form encapsulated for oral administration, the composition further comprises:
(a) a cellulose acetate phthalate (C-A-P) at a concentration from 5% to 15% by weight based on the total weight of the preparation;
(b) a solvent that includes polyethylene glycol, and in some embodiments, at a molecular weight from 200 to 600 and/or propylene carbonate, wherein the concentration of polyethylene glycol is from 50% to 80% by weight based on the total weight of the dosage form and the concentration of propylene carbonate is from 0% to 15% by weight based on the total weight of the dosage form; and
(c) triacetin, and in some embodiments, at a concentration from 0% to 30% of the C-A-P weight.

Certain liquid forms encapsulated for oral administration of this type can be found in WO 2007/050294 published May 3, 2007, which is incorporated by reference herein.

In some embodiments when the composition comprises a liquid solution, the composition further comprises at least one triglyceride selected from the group consisting of short chain triglycerides, medium chain triglycerides, and long chain triglycerides, wherein the composition has less than about 5% water (w/v).

Certain liquid solution formulations of this type can be found in US 2008/0076749 published Mar. 27, 2008, which is incorporated by reference herein.

In some embodiments when the composition comprises a liquid suspension, the composition comprises:
(a) from about 0.01% up to about 50% of the at least one therapeutic agent (w/w);
(b) from about 20 to about 64% water (w/w);
(c) up to about 50% glycerin (w/w);
(d) up to about 20% sorbitol (w/w);
(e) up to about 10% propylene glycol (w/w);
(f) up to about 3% surfactant (w/w); and
(g) up to about 1% of a thickening agent (w/w) 4.

In some embodiments when the composition comprises a liquid suspension, the composition comprises:
(a) from about 0.01% up to about 50% of the at least one therapeutic agent (w/w);
(b) from about 20 to about 64% water (w/w);
(c) up to about 50% glycerin (w/w);
(d) up to about 25% sorbitol (w/w);
(e) up to about 20% propylene glycol (w/w); and
(f) up to about 1% of a thickening agent (w/w) 4.

Certain liquid suspension formulations of this type can be found in WO 2003/105804 published Dec. 24, 2003, and WO 2005/076829 published Aug. 25, 2005, which are both incorporated by reference herein.

In some embodiments, the composition can be delivered using a spill-resistant delivery system, said system comprises
(a) a squeezable container having an outlet defining a flow channel;
(b) a dispenser connected to the container; and
(c) at least one dose of the composition.

Certain spill-resistant delivery system of this type can be found in U.S. Pat. No. 5,881,926 issued Mar. 16, 1999, and U.S. Pat. No. 6,102,254 issued Aug. 15, 2000, which are both incorporated by reference herein.

Additional spill-resistant delivery systems or spill-resistant formulations of this type can be found in U.S. Pat. No. 6,355,258 issued Mar. 12, 2002, U.S. Pat. No. 6,399,079 issued Jun. 4, 2002, U.S. Pat. No. 6,656,482 issued Dec. 2, 2003, US 2002/0168411 published Nov. 14, 2002, US 2003/0235618 published Dec. 25, 2003, WO 99/62498 published Dec. 9, 1999, WO 2003/034991 published May 1, 2003, WO 2007/117605 published Oct. 18, 2007, which are all incorporated by references herein.

In some embodiments, the composition of the method provided comprises:
(a) sugar spheres;
(b) an active substance layer comprising the at least one therapeutic agent and one or more water-soluble auxiliaries;
(c) a first lacquer layer, in some embodiments, comprising 80 to 97% of at least one lacquer which is insoluble in gastric fluid and soluble in intestinal fluid and 3 to 20% of at least one lacquer which is insoluble in gastric and intestinal fluids; and,
(d) a second lacquer, in some embodiments, consisting of 100% of at least one lacquer which is insoluble in gastric and intestinal fluids.

Certain compositions of this type can be found in U.S. Pat. No. 5,932,249 issued Aug. 13, 1999, which is incorporated by reference herein.

In some embodiments, the composition of the method provided comprises:
(a) an inner lipophilic matrix, in some embodiments, consisting of substances selected from the group consisting of unsaturated and/or hydrogenated fatty acid, salts, esters or amides thereof, fatty acid mono-, di- or triglycerides, waxes, ceramides, and cholesterol derivatives with melting points below 90° C., and wherein the active ingredient is dispersed both in said the lipophilic matrix and in the hydrophilic matrix;
(b) an outer hydrophilic matrix, in some embodiments, wherein the lipophilic matrix is dispersed, and said outer hydrophilic matrix consists of compounds selected from the group consisting of polymers or copolymers of acrylic or methacrylic acid, alkylvinyl polymers, hydroxyalkyl celluloses, carboxyalkyl celluloses, polysaccharides, dextrins, pectins, starches and derivatives, alginic acid, and natural or synthetic gums;
(c) optionally other excipients;
wherein, in some embodiments, the active ingredient is present in an amount of 80 to 95% by weight of the total composition, and wherein the active ingredient is dispersed both in the lipophilic matrix and in the hydrophilic matrix.

Certain compositions of this type can be found in U.S. Pat. No. 6,773,720 issued Aug. 10, 2004, which is incorporated by reference herein.

In some embodiments, the composition of the method provided comprises:
(a) a lipophilic matrix, in some embodiments, consisting of lipophilic compounds with a melting point between 40° C. and 90° C. in which the active ingredient is at least partially inglobated;
(b) an amphiphilic matrix;
(c) an outer hydrophilic matrix, in some embodiments, consisting of hydrogel forming compounds in which the lipophilic matrix and the amphiphilic matrix are dispersed, wherein the combination of the matrices from (a), (b), and (c) provides controlled release.

Certain compositions of this type can be found in U.S. Pat. No. 7,410,651 issued Aug. 12, 2008, and US 2009/0011010 published Jan. 8, 2009, which are both incorporated by reference herein.

In some embodiments, the composition of the method provided comprises:
(a) a matrix, comprising at least partially dispersed the at least one therapeutic agent, at least one wax, and at least one saturated fatty acid or unsaturated fatty acid;
(b) a hydrophilic matrix in which the lipophilic matrix is dispersed, said hydrophilic matrix consisting of carboxyalkylcellulose; and
(c) a matrix consisting of amphiphilic compounds, in some embodiments, being selected from the group consisting of polar lipids of type I, polar lipids of type II and glycols partially etherified with C₁-C₄ alkyl chains, in which the active ingredient is at least partially dispersed, said matrix of amphiphilic compounds being dispersed in the hydrophilic matrix,
wherein, in some embodiments, the matrix of the at least one therapeutic agent, the matrix of hydrophilic compounds, and the matrix of amphiphilic compounds, in combination, provide controlled release.

Certain compositions of this type can be found in U.S. Pat. No. 7,410,652 issued Aug. 12, 2008, which is incorporated by reference herein.

In some embodiments, the composition of the method provided comprises:
a hydrophilic first matrix comprising a lipophilic phase and an amphiphilic phase,
wherein said lipophilic phase and said amphiphilic phase are in a second matrix together, and said second matrix is dispersed throughout the hydrophilic first matrix,
wherein said hydrophilic first matrix, in some embodiments, consists of compounds selected from the group consisting of acrylic or methacrylic acid polymers, acrylic copolymers, methacrylic copolymers, alkyl vinyl polymers, hydroxyalkylcellulose, carboxyalkylcellulose, polysaccharides, dextrins, pectines, starches, starch derivatives, alginic acid, natural gums, synthetic gums, and polyalcohols,
wherein said lipophilic phase, in some embodiments, is in a granular form and, in some embodiments, consists of compounds with a melting point between 40 and 90° C. and an active ingredient at least partially incorporated in said lipophilic phase,
wherein said amphiphilic phase comprises an active ingredient at least partially incorporated in said amphiphilic phase.

Certain compositions of this type can be found in U.S. Pat. No. 7,431,943 issued Aug. 10, 2008, and US 2003/0165485 published Sep. 4, 2003, which are both incorporated by reference herein.

Additional compositions and methods of administration of this type can be found in U.S. Pat. No. 7,452,872 issued Nov. 18, 2008, US 2007/0265232 published Nov. 15, 2007, US 2008/0096849 published Apr. 24, 2008, WO 2007/025146 published Mar. 1, 2007, and WO 2008/063211 published May 29, 2008, which are all incorporated by reference herein.

In some embodiments, the composition of the method provided further comprises:
a pharmaceutically acceptable amine having a pK of about 8 or greater,
wherein the molar ratio of amine/the at least one therapeutic agent is at least about 5:1.

Certain compositions of this type can be found in US 2007/0020187 published Jan. 25, 2007, and WO 2007/009806 published Jan. 25, 2007, which are both incorporated by reference herein.

In some embodiments, the composition of the method provided further comprises:
at least one short-chain fatty acid or salt, ester and/or amide thereof, in combination with a complex sugar and/or dietary fiber in which the complex sugar and/or dietary fiber is selected from inulin, pectin, dextrin, maltodextrin or derivatives thereof and with one or more pharmacologically acceptable excipients.

Certain compositions of this type can be found in US 2007/0128266 published Jun. 7, 2007, and WO 2005/074718 published Aug. 18, 2005, which are both incorporated by reference herein.

In some embodiments, the composition of the method provided comprises:
a multi-matrix structure comprising:
(a) an amphiphilic matrix in which the at least one therapeutic agent is incorporated;
(b) a lipophilic matrix which is formed by substances having a melting point of less than 90° C. and in which (a) is dispersed;
(c) a hydrophilic matrix.

Certain compositions of this type can be found in US 2007/0248664 published Oct. 25, 2007, US 2008/0233193 published Sep. 25, 2008, and WO 2006/003043 published Jan. 12, 2006, which are both incorporated by reference herein.

In some embodiments, the composition of the method provided further comprises:
(a) a bulking agent comprises mannitol, lactose or mixture thereof;
(b) a binder selected from the group consisting of polyvinylpyrrolidone, sodium carboxymethylcellulose, microcrystalline cellulose, and mixture thereof;
(c) a disintegrating agent selected from the group consisting of maize starch, sodium starch glycolate, microcrystalline cellulose, sodium croscarmellose, calcium or sodium carboxymethylcellulose, crospovidone, and mixture thereof.

Certain compositions of this type can be found in US 2008/0206327 published Aug. 28, 2008, which is incorporated by reference herein.

In some embodiments, the composition of the method provided further comprises:
(a) at least one soluble binder comprising polyethylene glycol;
(b) at least one bulk-forming purgative comprises methylcellulose, sodium carboxymethyl cellulose, bran, psyllium, sterculia, and/or testa ispaghula.

Certain compositions of this type can be found in US 2008/0213393 published Sep. 4, 2008, which is incorporated by reference herein.

In some embodiments, the composition of the method provided further comprises:
a therapeutically effective amount of polyethylene glycol having an average molecular weight of at least 4000 and optional electrolytes.

Certain compositions of this type can be found in US 2008/0260682 published Oct. 23, 2008, and WO 2006/122104 published Nov. 16, 2006, which are both incorporated by reference herein.

In some embodiments, the composition of the method provided further comprises an ointment preparation comprising rifaximin.

Certain compositions of this type can be found in WO 2007/103448 published Sep. 13, 2007, and WO 2008/016708 published Feb. 7, 2008, which are both incorporated by reference herein.

In some embodiments, the composition of the method provided further comprises an acid and sugars or polyalcohols or a mixture thereof characterized in that at least one sugar or one polyalcohol has a negative temperature of dissolution.

Certain compositions of this type can be found in WO 2007/144323 published Dec. 21, 2007, which is incorporated by reference herein.

In some embodiments, the composition of the method provided comprises
(a) a substantially water insoluble bound bioactive agent complex, and in some embodiments, consisting essentially of the bioactive agent bound to an ion exchange resin and
(b) an aqueous carrier compatible with the bioactive agent, and in some embodiments, consisting essentially of water and a bulk-forming agent selected from the group consisting of gelatin, polyvinylpyrrolidone, polyethylene glycol, polysaccharides, and combinations thereof.

Certain compositions of this type can be found in U.S. Pat. No. 5,188,825 issued Feb. 23, 1993, which is incorporated by reference herein.

In some embodiments, the composition of the method provided is prepared by a process comprising the steps of
(a) blending the at least one therapeutic agent with a moisture absorber, in some embodiments, in the range of 0.5 to 20% by weight of total composition and a disintegrating agent, in some embodiments, in the range of 0.5 to 15% of the total composition and
(b) compacting the blend to obtain granules.

Certain compositions of this type can be found in WO 03/086343 published Oct. 23, 2003, which is incorporated by reference herein.

In some embodiments, the composition of the method provided further comprises:
coarse particles comprising the at least one therapeutic agent, and
in some embodiments, said coarse particles having a size within the range of 50 μm to 400 μm, and in some embodiments, which are coated with a polymer or lipid material in a matrix comprising a carrier material and a structure forming agent, and
in some embodiments, wherein said carrier material is selected from the group consisting of water soluble and water-dispersible carrier materials, and
in some embodiments, wherein said dosage form disintegrates in water in less than 10 seconds.

Certain compositions of this type can be found in U.S. Pat. No. 5,976,577 issued Nov. 2, 1999, and U.S. Pat. No. 6,413,549 issued Jul. 2, 2002, which are both incorporated by reference herein.

In some embodiments, the composition of the method provided comprises

a plurality of highly plastic granules, said granules comprising
(a) a porous, plastic substance, (b) a water penetration enhancer, and (c) a binder.

Certain compositions of this type can be found in WO 2004/100857 published Nov. 25, 2004, which is incorporated by reference herein.

In some embodiments when the composition comprises a hard, compressed, rapidly dissolvable dosage form adapted for direct oral dosing, the composition further comprises
a matrix including a non-direct compression filler and a lubricant; in some embodiments, said dosage form being adapted to rapidly dissolve in the mouth of a patient and thereby liberate said active ingredient, and in some embodiments, having a friability of about 2% or less when tested according to the U.S.P.

Certain formulations of this type can be found in U.S. Pat. No. 6,221,392, which is incorporated by reference herein.

In some embodiments when the composition comprises a solid pharmaceutical dosage form adapted for direct oral administration to a human comprising:
(a) a mixture of at least one saliva activated effervescent agent and
(b) a plurality of microparticles (including nanoparticles), in some embodiments, each said microparticle (including a nanoparticle) including at least one systemically distributable pharmaceutical ingredient and a protective material substantially encompassing said pharmaceutical ingredient,
in some embodiments, said microparticles (including nanoparticles) having at least about 70 percent 30-minute release, said mixture being present as a tablet of a size, and in some embodiments, shape adapted for direct oral administration to a human patient, in some embodiments, said tablet being substantially completely disintegrable so as to release said microparticles (including nanoparticles) upon exposure to saliva, and in some embodiments, said at least one effervescent agent being present in an amount which is effective to aid in rapid disintegration of said tablet, without chewing, and thereby release said microparticles (including nanoparticles).

In other embodiments when the composition comprises a solid pharmaceutical dosage form adapted for direct oral administration to a human comprising:
(a) a mixture of at least one saliva activated effervescent agent and
(b) a plurality of microparticles (including nanoparticles),
in some embodiments, each said microparticle (including a nanoparticle) including at least one systemically distributable pharmaceutical ingredient and a protective material substantially encompassing said pharmaceutical ingredient, and in some embodiments, said microparticles (including nanoparticles) being susceptible to release of said pharmaceutical ingredient upon rupture thereof, said mixture being present as a tablet of a size and shape adapted for direct oral administration to a human patient, and in some embodiments, said tablet being substantially completely disintegrable so as to release said microparticles (including nanoparticles) upon exposure to saliva, and in some embodiments, said at least one effervescent agent being present in an amount which is effective to aid in rapid disintegration of said tablet, without chewing, and thereby release said microparticles (including nanoparticles).

Certain formulations of this type can be found in U.S. Pat. No. 5,178,878, which is incorporated by reference herein.

In some embodiments when the composition comprises a plurality of hard, compressed, rapidly dissolvable dosage forms adapted for direct oral dosing, the composition further comprises:
(a) a non-direct compression sugar or sugar alcohol, and (b) a lubricant,
in some embodiments, said dosage form being adapted to rapidly dissolve in the mouth of a patient and thereby liberate said active ingredient, and in some embodiments, having a friability of about 2% or less when tested according to the U.S.P., said dosage forms having a hardness of about 15 to about 50 Newtons.

Certain disintegrating tablet formulations of this type can be found in U.S. Pat. No. 6,024,981, which is incorporated by reference herein.

In some embodiments when the composition comprises a particular tableting aid, the composition further comprises:
particulate magnesium carbonate having adsorbed thereon at least one oil in an amount effective to produce a dry, free-flowing, particulate tableting aid; and
in some embodiments, wherein said particulate tableting aid is present in an amount greater than zero and less than about 20 percent by weight of the total composition.

Certain disintegrating tablet formulations of this type can be found in U.S. Pat. No. 5,401,513, which is incorporated by reference herein.

In some embodiments when the composition comprises a stabilized effervescent dosage form, the composition further comprises a particulate effervescent couple,
in some embodiments, said effervescent couple consisting essentially of a solid core of an edible acid and a coating of an edible base, and in some embodiments, the amount of said base used in forming said coating being less than a stoichiometric amount relative to said edible acid, in some embodiments, said edible acid core and said edible base coating being reacted such that at least some free unreacted edible acid remains, and in some embodiments, wherein said coating of said base retards reaction between said edible acid and said acid sensitive pharmaceutically active agent which would lessen the activity of said dosage form; and in some embodiments, said dosage form having an acid neutralization capacity of less than about 5.0.

Certain disintegrating tablet formulations of this type can be found in U.S. Pat. No. 5,503,846, which is incorporated by reference herein.

In some embodiments when the composition comprises a dosage form which disintegrates in the mouth of a patient, the composition comprises
(a) the at least one therapeutic agent in the form of a powder or microcapsule and, in some embodiments, in an amount which is sufficient to elicit therapeutic response;
(b) a filler; and
(c) at least one in-mouth viscosity enhancing material, in some embodiments, in an amount which is effective to provide an organoleptically acceptable viscous slurry having a viscosity range from between about 25,000 and about 500,000 CPS upon the disintegration of the dosage form in a patients mouth.

In some embodiments, the in-mouth viscosity enhancer can include gels, in-situ gel formers, gums and polymeric materials including are methylcellulose and hydroxypropylmethyl cellulose. In some embodiments, these materials are provided in an amount which is sufficient to increase the viscosity of the slurry that results from the disintegration and dissolution of the various other components of the tablet in a patient's mouth. However, in some embodiments, the amount of such in-mouth viscosity enhancing ingredients can be controlled to ensure that an organoleptically acceptable slurry results and that the increased viscosity does not too adversely affect either the in-mouth disintegration time or the organoleptic properties of the formulation.

In other embodiments when the composition comprises an orally disintegrable tablet suitable for use in the delivery of at least one active ingredient in the form of microcapsules or powders, the composition comprises between about 10 and about 80% of the at least one therapeutic agent containing microcapsules or powders by weight based on the weight of the tablet,
in some embodiments, said microcapsules or powder having a particle size ranging from between about 50 to about 3,000 microns, and in some embodiments, an amount of at least one in-mouth viscosity enhancer, in some embodiments, which is sufficient to provide a viscous, swallowable, organoleptically acceptable mass containing said microcapsules, within about 3 minutes when placed in a patients mouth, and in some embodiments, said in-mouth viscosity enhancer being selected from the group consisting of methylcellulose, hydroxypropylmethyl cellulose, hydroxyethyl cellulose, carbopol and silicon dioxide; and in some embodiments, optionally between 0 and about 60% of a rapidly dissolvable sugar or sugar alcohol filler by weight of the tablet selected from the group consisting of sucrose, mannitol, xylitol, lactose and maltose; and in some embodiments, optionally between 0 and about 35% of a binder by weight of the tablet selected from the group consisting of microcrystalline cellulose and methyl cellulose; optionally between 0 and about 40% of a disintegrant by weight based on the weight of the tablet selected from the group consisting of sodium starch glycolate and crospovidone; and in some embodiments, optionally between 0 and about 50% of an effervescent couple based on the weight of the tablet, in some embodiments, wherein said effervescent couple is present and is present in an amount of between about greater than zero and about 50% by weight based on the weight of the finished dosage form.

Certain disintegrating tablet formulations of this type can be found in U.S. Pat. No. 6,368,625, which is incorporated by reference herein.

In some embodiments when the composition comprises a rapidly soluble tablet for oral administration of a therapeutic substance to a human or animal, the composition further comprises
a kneaded wet compressed shaped tablet which comprises an amount of a pharmaceutical additive, in some embodiments, which is rapidly soluble in water sufficient to form a rapidly soluble tablet, and in some embodiments, said tablet having been compression shaped with a compression force of from about 150 to about 1000 kg in a wet state and thereafter dried, and in some embodiments, said wet tablet containing from about 1 to about 10% by weight of water.

Certain disintegrating tablet formulations of this type can be found in U.S. Pat. No. 5,837,285, which is incorporated by reference herein.

In some embodiments when the composition comprises fast dissolving tablet obtainable by compression-molding, the composition further comprises
30 to 80% by weight of a water-soluble carbohydrate,
in some embodiments, having a mean particle size of 20 to 70 um and 1 to 3% by weight of water into a tablet form, in some embodiments, at a pressure of 5 to 130 Kg/cm2 and drying the compression-molded tablet, and in some embodiments, said fast dissolving tablet having (i) a porosity of 30 to 70%, (ii) a hardness of 3 to 20 kg, and (iii) a falling impact strength of 0 to 70%.

Certain disintegrating tablet formulations of this type can be found in U.S. Pat. No. 5,720,974, which is incorporated by reference herein.

In some embodiments when the composition comprises an intrabuccally dissolving compressed molding showing quick disintegration and dissolution in the buccal cavity, the composition comprises granules comprising a saccharide having low moldability, in some embodiments, having been granulated with a saccharide having high moldability.

Certain disintegrating tablet formulations of this type can be found in U.S. Pat. No. 5,576,014, which is incorporated by reference herein.

In some embodiments when the composition comprises a drug-containing particle, the composition comprises a particle, comprising the at least one therapeutic agent, has a mean particle diameter of approximately 50 to approximately 250 um and, in some embodiments, an apparent specific gravity of approximately 0.5 to approximately 1.2, and, in some embodiments, comprises a bitter tasting drug and a water-insoluble polymer.

Certain disintegrating tablet formulations of this type can be found in U.S. Pat. No. 6,656,492, which is incorporated by reference herein.

In some embodiments when the composition comprises a quick-disintegrating tablet in the buccal cavity, the composition further comprises
(a) lactose and/or mannitol, (b) erythritol, and (c) maltitol,
in some embodiments, wherein the tablet hardness is 3 kp or higher and the friability is 1% or less and, in some embodiments, porosity is approximately 30 to approximately 50%, or
(b) lactose and/or mannitol, (b) erythritol, and (c) copolyvidone,
in some embodiments, wherein the tablet hardness is 3 kp or higher and the friability is 1% or less and, in some embodiments, porosity is approximately 30 to approximately 50%.

Certain disintegrating tablet formulations of this type can be found in U.S. Pat. No. 6,872,405, which is incorporated by reference herein.

In some embodiments when the composition comprises

a cured comestible unit which can dissolve in the mouth in less than 10 seconds prepared by the method comprising:
(a) mixing a chopped uncured shearform matrix and an additive;
(b) compressing to a density of about 1.2 or less; and curing said compressed shearform matrix by subjecting it to conditions of heat, moisture and pressure which induce crystallization.

Certain disintegrating tablet formulations of this type can be found in U.S. Pat. No. 5,851,553, which is incorporated by reference herein.

In some embodiments when the composition comprises a quick dissolve comestible unit containing a controlled-release system, the composition comprising a unit comprising a molded tablet prepared by a method of: mixing uncured shearform matrix particles and a controlled-release system; in some embodiments, molding the mixture to yield a unit dosage form; and curing said mixture.

Certain disintegrating tablet formulations of this type can be found in U.S. Pat. No. 6,020,002, which is incorporated by reference herein.

In some embodiments when the composition comprises a tablet, the composition comprises a natural polymer or a hydrolysate of a natural polymer, or a mixture thereof, and in some embodiments, wherein the core is uncoated; or is partially or completely coated with no more than one layer, the layer comprising a lipid compound covalently bonded to the core, or an amphiphilic compound.

Certain disintegrating tablet formulations of this type can be found in U.S. Pat. No. 6,017,513, which is incorporated by reference herein.

In some embodiments when the composition comprises a tablet, the composition further comprises
(a) a low melting point compound that melts or softens at or below 37° C., and
(b) a water soluble excipient,
in some embodiments, wherein the low melting point compound comprises from about 2.5% to about 20% (wt/wt) of the composition, and wherein the tablet has a hardness of about 1 kilopond or lower.

Certain disintegrating tablet formulations of this type can be found in U.S. Pat. No. 6,733,781, which is incorporated by reference herein.

In some embodiments when the composition comprises a tablet, the composition further comprises
(a) a low melting point compound that melts or softens at or below 37° C., and
(b) a water soluble excipient,
in some embodiments, wherein the low melting point compound comprises from about 0.01% to about 2.5% (wt/wt) of the tablet, and wherein the tablet has a hardness of about 2.0 or lower.

Certain disintegrating tablet formulations of this type can be found in US Publication No. 2004/0037878, which is incorporated by reference herein.

In some embodiments when the composition comprises a water-dispersible tablet, the composition comprises
(a) microparticles which contain at least one pharmaceutically active substance
(b) at least one disintegrant and
(c) a swellable material which is able to generate a high viscosity when coming into contact with water and
in some embodiments, which is selected from the group consisting of guar gum, xanthan gum, alginates, dextran, pectins, polysaccharides, sodium or calcium carboxymethylcellulose, hydroxypropylcellulose and hydroxypropylmethylcellulose; in some embodiments, which tablet disintegrates rapidly in water forming a homogeneous suspension of high viscosity that can easily be swallowed.

Certain disintegrating tablet formulations of this type can be found in U.S. Pat. No. 4,886,669, which is incorporated by reference herein.

In some embodiments when the composition comprises a effervescent granule, in some embodiments, the composition comprises a mixture consisting essentially of an agent, the at least one therapeutic agent, a hot-melt extrudable binder and an alkaline agent; in some embodiments, the effervescent granule being made by an essentially water free, and in some embodiments, essentially solvent free thermal heat process comprising: dry blending said mixture; and in some embodiments, hot-melt extruding said blended mixture to form an effervescent granule.

Certain disintegrating tablet formulations of this type can be found in U.S. Pat. No. 6,071,539, which is incorporated by reference herein.

In some embodiments when the composition comprises an effervescent granule having a controllable rate of effervescence, the composition comprises a mixture consisting essentially of an agent, pharmacologically active agent, in some embodiments, a hot-melt extrudable binder and optionally a plasticizer; and an alkaline agent; in some embodiments, the effervescent granule made by an essentially water free and essentially solvent free thermal heat process comprising: dry blending said mixture; and hot-melt extruding said blended mixture to form an effervescent granule.

Certain disintegrating tablet formulations of this type can be found in U.S. Pat. No. 6,649,186, which is incorporated by reference herein.

In some embodiments when the composition comprises an orodispersible effervescent tablet, the composition further comprises
(a) at least one disintegrant, in some embodiments, selected from the group consisting of croscarmellose, crospovidone and mixtures thereof,
(b) a diluent soluble agent, and
(c) a lubricant, and
in some embodiments, effervescent granules based on at least one active principle and a mixture consisting essentially of an acidic agent, a heat-extrudable binder and an alkaline agent, and in some embodiments, optionally a dehydrating agent and a lubricant, in some embodiments, said granules being prepared by heat extrusion in the absence of water and solvents, and in some embodiments, said tablet undergoing disaggregation in the buccal cavity in contact with saliva in less than 60 seconds.

In other embodiments when the composition comprises an orodispersible effervescent tablet, the composition comprises
(a) at least one disintegrant selected from the group consisting of croscarmellose, crospovidone and mixtures thereof,
(b) a diluent soluble agent, and
(c) a lubricant, and
in some embodiments, effervescent granules based on at least one active principle and a mixture consisting essentially of an acidic agent, a heat-extrudable binder and an alkaline agent, and in some embodiments, optionally a dehydrating agent and a lubricant, said granules being prepared by heat extrusion in the absence of water and solvents, and in some embodiments, said tablet undergoing disaggregation in the buccal cavity in contact with saliva in less than 60 seconds.

Certain disintegrating tablet formulations of this type can be found in US Publication No. 2004/0265380, which is incorporated by reference herein.

In some embodiments, a composition comprises a medicament in a pharmaceutically acceptable effervescent formulation, said effervescent formulation comprising: at least one first gas contained within an aqueous dissolvable solid matrix, and in some embodiments, at least two components reactive to generate a second gas upon aqueous contact.

Certain disintegrating tablet formulations of this type can be found in US Publication No. 2005/0074489, which is incorporated by reference herein.

In some embodiments when the composition comprises a composition in the form of a solid carrier, the composition comprises a substrate and an encapsulation coat on the substrate, in some embodiments, wherein the encapsulation coat comprises an admixture of a therapeutically effective amount of a hydrophobic pharmaceutical active ingredient, an effective solubilizing amount of at least one hydrophilic surfactant, and a lipophilic additive selected from the group consisting of lipophilic surfactants, triglycerides, and combinations thereof, in some embodiments, wherein the effective solubilizing amount of the at least one hydrophilic surfactant is an amount effective to partially or fully solubilize the pharmaceutical active ingredient in the encapsulation coat.

Certain disintegrating tablet formulations of this type can be found in U.S. Pat. No. 6,569,463, which is incorporated by reference herein.

In some embodiments when the composition comprises a composition in the form of a solid carrier, the composition comprises a substrate and an encapsulation coat on the substrate, in some embodiments, wherein the encapsulation coat comprises an admixture of a therapeutically effective amount of a hydrophilic pharmaceutical active ingredient, in some embodiments, an effective solubilizing amount of at least one hydrophilic surfactant, and in some embodiments, a lipophilic additive selected from the group consisting of lipophilic surfactants, triglycerides, and combinations thereof, in some embodiments, wherein the effective solubilizing amount of the at least one hydrophilic surfactant is an amount effective to partially or fully solubilize the pharmaceutical active ingredient in the encapsulation coat.

Certain disintegrating tablet formulations of this type can be found in U.S. Pat. No. 6,248,363, which is incorporated by reference herein.

In some embodiments when the composition is for enhanced absorption of a hydrophilic therapeutic agent, the composition comprises a dosage form of an absorption enhancing composition, the composition comprising:
(a) at least one hydrophilic surfactant selected from the group consisting of ionized ionizable surfactants, non-ionic hydrophilic surfactants having an HLB value greater than or equal to about 10, and combinations thereof, and
(b) at least one hydrophobic surfactant, in some embodiments, selected from the group consisting of hydrophobic
   (i) alcohols, polyoxyethylene alkylethers, bile acids, glycerol fatty acid monoesters, glycerol fatty acid diesters, acetylated glycerol fatty acid monoesters, acetylated glycerol fatty acid diesters, lower alcohol fatty acid monoesters, lower alcohol fatty acid diesters, polyethylene glycol fatty acid esters, polyethylene glycol glycerol fatty acid esters, polypropylene glycol fatty acid esters, polyoxyethylene glycerides, lactic acid derivatives of mono- and diglycerides, propylene glycol diglycerides, sorbitan fatty acid esters, polyoxyethylene sorbitan fatty acid esters, polyoxyethylene-polyoxypropylene block copolymers, transesterified vegetable oils, sugar esters, sugar ethers, sucroglycerides, polyoxyethylene vegetable oils, polyoxyethylene hydrogenated vegetable oils, reaction products of polyols and at least one member of the group consisting of fatty acids, glycerides, vegetable oils, and hydrogenated vegetable oils, and hydrophobic, un-ionized
   (ii) fatty acids, carnitine fatty acid esters, alkylsulfates, acyl lactylates, mono-acetylated tartaric acid esters of mono- and diglycerides, diacetylated tartaric acid esters of mono- and diglycerides, succinylated monoglycerides, citric acid esters of mono- and diglycerides, and mixtures thereof, wherein the hydrophilic and hydrophobic surfactants are present in amounts such that upon mixing with an aqueous diluent at 100× dilution, in some embodiments, the composition forms a clear aqueous dispersion having an absorbance of less than about 0.3 at 400 nm; and
(c) a therapeutically effective amount of a hydrophilic therapeutic agent, in some embodiments, wherein the pharmaceutical system is free of triglycerides.

Certain disintegrating tablet formulations of this type can be found in U.S. Pat. No. 6,309,663, which is incorporated by reference herein.

In some embodiments when the composition comprises a dosage form, the composition comprises
(a) the at least one therapeutic agent having a first fraction and a second fraction, in some embodiments, wherein the first fraction is comprised of a plurality of solid particles; and
(b) a pharmaceutically acceptable vehicle comprising at least one compound,
in some embodiments, selected from the group consisting of a hydrophilic surfactant, a lipophilic surfactant, a triglyceride and a solubilizer, wherein the first fraction of the active agent is suspended in the vehicle and the second fraction of the active agent is solubilized in the vehicle, in some embodiments, said first fraction representing about 5 wt. % to about 80 wt. % of the active agent and in some embodiments, said second fraction representing about 20 wt. % to about 95 wt. % of the active agent.

Certain disintegrating tablet formulations of this type can be found in US Publication No. 2003/0077297, which is incorporated by reference herein.

In some embodiments when the composition comprises a composition in the form of a solid carrier, the composition comprises an admixture of: the at least one therapeutic agent, and at least one hydrophilic surfactant, in some embodiments, wherein the hydrophilic surfactant is selected from the group consisting of: lauryl macrogolglycerides; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; tocopherol polyethylene glycol succinates; sugar esters; fatty acid derivatives of amino acids, carnitines, oligopeptides, and polypeptides; glyceride derivatives of amino acids, oligopeptides, and polypeptides; acyl lactylates; mono-, diacetylated tartaric acid esters of mono-, diglycerides; succinylated monoglycerides; citric acid esters of mono-, diglycerides; alginate salts; propylene glycol alginate; lecithins and hydrogenated lecithins; salts of alkylsulfates; salts of fatty acids; sodium docusate; and mixtures thereof.

Certain disintegrating tablet formulations of this type can be found in US Publication No. 2006/0034937, which is incorporated by reference herein.

In some embodiments when the composition comprises a solidified buccal or transmucosal oral mucosa adherent dosage form having dimensions which fit into the buccal cavity or under the tongue of a user thereof, the composition the at least one therapeutic agent adapted to be dispensed typically over a period of 10-30 minutes through transmucosal absorption directly into the bloodstream, in some embodiments, said pharmaceutical ingredient being dispersed in a non-crystalline, solidified polymeric matrix which adheres to oral mucosa after being activated by water or saliva comprising, from about 20 to about 75 percent by weight of a polyethylene glycol component having a low molecular weight of from about 100-4000, in some embodiments, from about 2 to about 54 percent by weight of a polyethylene glycol component having a medium to high molecular weight of from about 6000-20,000, and in some embodiments, from about 1 to about 40 percent by weight of polyethylene oxide having a high molecular weight of from about 100,000 to 5,000,000.

Certain disintegrating tablet formulations of this type can be found in U.S. Pat. No. 4,764,378, which is incorporated by reference herein.

In some embodiments when the composition comprises a buccal dosage forms with polyethylene glycols, the composition comprises
(a) Low MW Polyethylene glycol 75-90% (M.P. about 37° C.);
(b) Medium to high MW polyethylene glycol 0-4%;
(c) Polyethylene oxide (MW 100 k-5 Mk) 0.1-4%;
(d) Colloidal silica 10-20%; and optionally,
(e) Long chain saturated carboxylic acid 0.5-4%.

Certain disintegrating tablet formulations of this type can be found in U.S. Pat. Nos. 5,244,668 and 5,139,790, which is incorporated by reference herein.

In some embodiments when the composition comprises a chemical complex of an active ingredient and an aminosugar.

Certain disintegrating tablet formulations of this type can be found in US Patent No. 2005/0130935, which is incorporated by reference herein.

In some embodiments when the composition comprises a flowable composition for comestible units, the composition comprises
(a) partially crystallized shearform particles treated with at least one of ethanol and lactose, and
(b) particles containing the at least one therapeutic agent.

Certain disintegrating tablet formulations of this type can be found in U.S. Pat. No. 6,048,541, which is incorporated by reference herein.

In some embodiments when the composition comprises a flowable particulate composition for dosage units, the composition comprises
(a) at least one flowable, partially crystallized amorphous matrix, in some embodiments, said matrix comprising at least one carbohydrate and at least one sugar alcohol;
(b) a bio-affecting agent, in some embodiments, in the form of a coated or uncoated particle; and
(c) a tableting aid.
In one non-limiting example, the at least one matrix comprises one flowable, partially crystallized amorphous matrix comprising at least one carbohydrate and a mixture of sugar alcohols.

In other embodiments when the composition comprises rapid dissolve tablet which dissolves in the mouth, with or without water, in from about 3 seconds to about 30 seconds, the composition comprises
(a) a crystallized matrix comprising a carbohydrate and at least one sugar alcohol; and
(b) an active agent.

Certain disintegrating tablet formulations of this type can be found in U.S. Pat. No. 5,869,098, which is incorporated by reference herein.

In some embodiments, the composition comprises a porous matrix which comprises a wetting agent and microparticles (including nanoparticles) of a drug, wherein the microparticles (including nanoparticles) a mean diameter between about 0.1 and 5 μm, and in some embodiments, a total surface area greater than about 0.5 m2/mL, and in some embodiments, wherein the porous matrix has a TAP density less than or equal to 1.0 g/mL and/or has a total surface area of greater than or equal to 0.2 m2/g, and in some embodiments, in the form of a powder.

Certain disintegrating tablet formulations of this type can be found in U.S. Pat. No. 6,645,528, which is incorporated by reference herein.

In some embodiments, the composition comprises a therapeutically or prophylactically effective amount of the drug in a formulation comprising a porous matrix which comprises a wetting agent and microparticles (including nanoparticles) of the drug, in some embodiments, wherein the microparticles (including nanoparticles) have a mean diameter between about 0.1 and 5 μm and a total surface area greater than about 0.5 m²/mL, and in some embodiments, wherein the porous matrix has a TAP density less than or equal to 1.0 g/mL or has a total surface area of greater than or equal to 0.2 m²/g and is in the form of a dry powder, and in some embodiments, wherein the porous matrix is made by a process comprising, dissolving the drug in a volatile solvent to form a drug solution, combining at least one volatile salt with the drug solution to form an emulsion, suspension, or second solution, incorporating at least one wetting agent into the emulsion, suspension, or second solution, and in some embodiments, removing the volatile solvent and volatile salt from the emulsion, suspension, or second solution to yield the porous matrix.

Certain disintegrating tablet formulations of this type can be found in U.S. Pat. No. 6,932,983, which is incorporated by reference herein.

In some embodiments, the composition comprises a porous matrix of drug made by
(a) dissolving a drug in a volatile organic solvent to form a drug solution,
(b) combining at least one volatile pore forming agent with the volatile organic drug solution to form an emulsion, suspension, or second solution, and
(c) removing the volatile organic solvent and volatile pore forming agent from the emulsion, suspension, or second solution to yield the porous matrix comprising drug, in some embodiments, wherein the porous matrix comprising drug has a tap density of less than or equal to 1.0 g/mL or a total surface area of greater than or equal to 0.2 m²/g.

Certain disintegrating tablet formulations of this type can be found in U.S. Pat. No. 6,395,300, which is incorporated by reference herein.

In some embodiments, the composition comprises a porous matrix formed of a hydrophilic or hydrophobic excipient and microparticles (including nanoparticles) of a drug, in some embodiments, wherein the microparticles (including nanoparticles) have a mean diameter between about 0.1 and 5 μm and a total surface area greater than about 0.5 m2/mL, in some embodiments, wherein the dry porous matrix is in a dry powder form having a TAP density less than or equal to 1.0 g/mL, and in some embodiments, having a total surface area of greater than or equal to 0.2 m²/g.

Certain disintegrating tablet formulations of this type can be found in US Publication No. 2005/0048116, which is incorporated by reference herein.

In some embodiments, the composition comprises a therapeutically or prophylactically effective amount of a drug in a formulation comprising at least one hydrophilic or hydrophobic excipient and microparticles (including nanoparticles) of a drug, in some embodiments, wherein the microparticles (including nanoparticles) have a mean diameter between about 0.1 and 5 μm and a total surface area greater than about 0.5 m²/mL, and in some embodiments, wherein the dry porous matrix is in a dry powder form having a TAP density less than or equal to 1.0 g/mL, and in some embodiments, having a total surface area of greater than or equal to 0.2 m²/g.

Certain disintegrating tablet formulations of this type can be found in US Publication No. 2005/0058710, which is incorporated by reference herein.

In some embodiments, the composition comprises a porous matrix formed of at least one hydrophilic or hydrophobic excipient and microparticles (including nanoparticles) of a drug, in some embodiments, wherein the microparticles (including nanoparticles) have a mean diameter between about 0.1 and 5 μm and a total surface area greater than about 0.5 m²/mL, and wherein the dry porous matrix is in a dry powder form having a TAP density less than or equal to 1.0 g/mL and having a total surface area of greater than or equal to 0.2 m²/g, comprising
(a) dissolving a drug in a volatile solvent to form a drug solution,
(b) combining at least one pore forming agent with the drug solution to form an emulsion, suspension, or second solution,
(c) incorporating at least one excipient into the emulsion, suspension, or second solution, in some embodiments, wherein the excipient is selected from the group consisting of hydrophobic and hydrophilic excipients which enhance dissolution rate, in some embodiments, which stabilize drug in amorphous form by preventing crystallization, and which stabilize drug in crystalline form by inhibiting crystal growth, and
(d) removing the volatile solvent and pore forming agent from the emulsion, suspension, or second solution to yield the porous matrix of drug and excipient.

Certain disintegrating tablet formulations of this type can be found in US Publication No. 2002/0142050, which is incorporated by reference herein.

In some embodiments, the composition of the method provided is prepared by a process comprising the step of
(a) mixing the at least one therapeutic agent with a solvent, for example water, and a pharmaceutically acceptable polymer; and
(b) drying to form a solid composition,

in some embodiments, wherein said polymer is at least partially water soluble.

Certain compositions of this type can be found in U.S. Pat. No. 6,168,805 issued Jan. 2, 2001, which is incorporated by reference herein.

In some embodiments, the composition of the method provided comprises
(a) a first matrix including a first therapeutic agent;
(b) a second matrix including a second therapeutic; and
(c) a coating including a third therapeutic agent;
in some embodiments, wherein the composition is adapted to release at least about 30% of the second and the third therapeutic agents in the first hour based on dissolution according to USP XXIV Apparatus I, basket method at 100 rpm using 0.1 N HCl as dissolution medium at 37.5° C.

Certain compositions of this type can be found in US 2003/0004177 published Jan. 2, 2003, which is incorporated by reference herein.

In some embodiments, the composition of the method provided further comprises
an abuse deterring substance, for example a capsaicinoid,
in some embodiments, wherein said composition is for subsequent formulation into a final dosage form selected from a solid oral dosage form and a transdermal dosage form; and in some embodiments, wherein said capsaicinoid is present in an amount such that said final dosage form contains an amount effective to cause at least one response selected from coughing, sneezing, secretion, and pain when contacted with a mucosal or vascular membrane.

Certain compositions of this type can be found in US 2003/0064122 published Apr. 3, 2003, which is incorporated by reference herein.

In some embodiments, the composition of the method provided comprises:
(a) a controlled release matrix;
(b) a first therapeutic agent; and
(c) a second therapeutic agent,
in some embodiments, wherein said controlled release matrix is selected and incorporated for controlling the release rate of the second therapeutic agent;
in some embodiments, the controlled release matrix controls the release rate of both the first and the second therapeutic agents; and
in some embodiments, the composition remains substantially intact when orally administered to a patient.

Certain compositions of this type can be found in US 2003/0065002 published Apr. 3, 2003, and US 2008/0069881 published Mar. 20, 2008, which are both incorporated by reference herein.

In some embodiments, the composition of the method provided comprises
a controlled release matrix, and in some embodiments, from about 5 mg to about 80 mg the at least one therapeutic agent,
in some embodiments, wherein said composition provides a maximum blood concentration of the at least one therapeutic agent of between about 0.1 ng/ml and about 7.5 ng/ml upon oral administration of a single dose to a subject;
in some embodiments, wherein upon placement of the composition in an in vitro dissolution test comprising USP Paddle Method at 50 rpm in 500 ml media having a pH of 1.2 to 6.8 at 37° C., about 15% to about 50%, by weight, of the at least one therapeutic agent is released from the tablet after about 1 hour in the test;
in some embodiments, the composition has a release rate profile designed to provide an adequate blood plasma level over at least 12 hours to provide desired therapeutic effects; and in some embodiments, wherein the Cₘₐₓ is at least about 50% higher when the dosage form is administered to the subject under fed as compared to fasted conditions.

Certain compositions of this type can be found in US 2003/0157167 published Aug. 21, 2003, US 2007/0134328 published Jun. 14, 2007, and US 2008/0262013 published Oct. 23, 2008, which are all incorporated by reference herein.

In certain embodiments, a composition is provided in the form of a chewing gum. Chewing gums may contain any suitable amount of active. In certain embodiments, chewing gums comprise anywhere from 0.1 mg to as much as 1 g, or more, of the active agents. In some embodiments, the chewing gum is chewed over a time period that releases, for example, a corticosteroid in the month, thus reaching and coating the esophagus. In various embodiments, the chewing gum, when administered and chewed, provides for slow release and delivery of corticosteroid to the esophagus. In other embodiments, the chewing gum, when administered and chewed, provides for rapid release and delivery of corticosteroid to the esophagus. In other illustrative embodiments, the chewing gum comprises a liquid compartment or liquid center filled with corticosteroid described herein that breaks open upon chewing, thus reaching and coating the esophagus. Chewing gums include any shape or size, including but not limited to sticks, slabs, pellets, spheroids, chiclets, and the like.

Chewing gums compositions are formulated by any suitable method. In specific embodiments, such compositions comprise a chewable gum base and, optionally, one or more of flavoring agents, sweeteners, elastomers, colorants, preservatives, softeners, fillers/texturizers, and/or coatings. Other non-limiting examples of suitable chewing gum composition are described in U.S. Pat. No. 6,627,234; U.S. Pat. No. 6,586,023; U.S. Pat. No. 6,602,518; U.S. Pat. No. 6,592,850; U.S. Pat. No. 6,613,346; U.S. Pat. No. 6,558,692; U.S. Pat. No. 6,531,114; U.S. Pat. No. 6,465,003; U.S. Pat. No. 6,426,090; U.S. Pat. No. 6,355,265; U.S. Pat. No. 6,350,480; U.S. Pat. No. 6,322,806; and U.S. Pat. No. 6,290,985; each of which are incorporated by reference for such disclosures.

In other embodiments, the formulation may include medicinal foam (e.g., for oral administration to a patient). In certain instances, a medicinal foam is a gas-trapped liquid formulation that is easy to ingest and may provide for an alternative administration for those who have difficulty swallowing a viscous liquid or a solid oral dosage form. In certain embodiments, medicinal foams contain any suitable amount of active agent, e.g., 0.25 mg to 1 g of the active agents (or any amount described herein). In some embodiments, the medicinal foam is an aqueous, alcohol, or oil based composition that is aerosolized. In certain embodiments, the medicinal foam is a liquid formulation as described herein that is aerosolized. In some embodiments, suitable liquid bases for medicinal foams include, but are not limited to, oils and fatty acids such as soybean oil, partially hydrogenated soybean oil, linseed oil, corn oil, peanut oil, sunflower oil, cottonseed oil, olive oil, liquid petrolatums, oleic acid, lauric acid and mono- and diglyceride oils, mineral oil, castor oil, fish liver oils, and fish body oils; water, ethanol, water/ethanol mixtures, water/oil mixtures and combinations thereof. In some embodiments, medicinal foams contain propellants to aerosolize the liquid composition and one or more sweeteners, foaming agents, preservatives, and taste masking agents.

Non-limiting examples of foaming agents include lecithins, polyol fatty acid esters such as glycerol esters of fatty acids (glycerol monostearate, glycerol monooleate, and the like), polyglycerol esters of fatty acids (hexaglycerol distearate, decaglycerol tetraoleate, triglycerol monostearate, triglycerol monooleate, octaglycerol monostearate, octaglycerol monooleate, and the like), sorbitan esters of fatty acids (sorbitan monostearate, sorbitan monooleate, sorbitan monopalmitate, and the like); proteins and protein hydrolyzates (caseins and caseinates, whey, gelatin, albumens, and mixtures thereof); cellulosic derivatives (methyl cellulose, hydroxymethyl cellulose, hydroxypropylmethylcellulose, hydroxypropyl cellulose, and the like), gums (xanthan gum, agar, carrageenan, guar gum, locust bean gum, and the like), and combinations thereof. In various embodiments, foaming agents are used in any suitable amount, e.g., from about 2% to about 40%, from about 3% to about 25%, or from about 5% to about 15% by weight of the medicinal foam.

In certain instances, taste masking agents suitable for medicinal foams include, but are not limited, to magnesium aluminum silicate, magnesium trisilicate, calcium carbonate, calcium silicate, a co-dried gel of aluminum hydroxide and magnesium carbonate, magnesium carbonate, ground limestone, ground oyster shells, and mixtures thereof.

In some instances, propellants are used to aerosolize the liquid pharmaceutical composition and to generate a medicinal foam and are generally odorless and tasteless. Suitable propellants are gaseous under atmospheric pressures and liquified when compressed. Exemplary propellants for medicinal foams include, by way of non-limiting example, propane, butane, isobutane, nitrogen, nitrous oxide, carbon dioxide, FREON 115, dichlorodifluoromethane, 1,1,1,2-tetrafluoroethane (HFC-134a). 1,1,1,2,3,3,3-heptafluoropropane (HFC-227), and mixtures thereof. In certain embodiments, the propellant is optionally utilized in any suitable amount, e.g., from about 5% to about 50%, from about 10% to about 40%, or from about 15% to about 25%.

In certain embodiments, medicinal foam compositions also encompass, as an article of manufacture, a pressurized aerosol container. Any suitable pressurized aerosol container can be used to administer medicinal foams. Various pressurized aerosol containers include single or multiple dose devices, metering systems, attached applicators and accessories, and other known dispensing elements to produce a stable medicinal foam.

In other illustrative embodiments, any of the compositions disclosed herein are provided in the form of a lozenge which may be dissolved in the mouth, thus reaching and coating the esophagus, and thereafter deliver the composition to the affected areas, including by way of example only, the lower esophagus, the esophageal-stomach juncture, the stomach, the duodenum and/or within 3 cm of the Z-line. The lozenge or other similar tablet, capsule, or other solid, would dissolve in the mouth or esophagus to produce a solution that can then coat the esophagus, and thereafter deliver the composition to the affected areas, including by way of example only, the lower esophagus, the esophageal-stomach juncture, the stomach and/or the duodenum. Or, for children, infants or other patients that may have difficulty with a dissolving lozenge, the lozenge may be ground or otherwise dissolved in a small volume of water or other pharmaceutically suitable liquid, for example, reaching a total volume presented in embodiments herein. In some embodiments, the lozenge or other similar tablet, capsule, or other solid is effervescent. In other illustrative embodiments of the invention, the compositions disclosed herein are provided in the form of a tablet, a capsule, or, for example a gel capsule, designed for slow release and delivery to the gastrointestinal tract, including the esophagus.

In some embodiments, the lozenge or other similar tablet, capsule, or other solid (e.g., powder) is effervescent. In certain embodiments, an effervescent dosage form is orally administered, delivering the dosage form to the stomach, whereupon the dosage form effervesces and provides delivery of the active(s) and optional excipients (e.g., coating agents, viscosity enhancing agents and/or mucoadhesive agents) to the esophagus. In certain embodiments, the effervescent dosage is a liquid dosage form or solid dosage form (e.g., a powder or tablet) dissolved in a small volume of water or other pharmaceutically suitable liquid to form a liquid composition. In such embodiments, the liquid composition can coat the esophagus, and thereby deliver the composition to the affected areas, including by way of example only, the esophagus, the lower esophagus, the esophageal-stomach juncture, the stomach and/or the duodenum. In some instances, the liquid composition is effervescent. The effervescent properties aid in administering the drug to the effective areas such as the esophagus or stomach.

In certain embodiments, the effervescent solid form and powders contains any suitable amount of active agent, e.g., 0.25 mg to 1 g of the active agents (or any amount described herein). Suitable volumes of water or other pharmaceutically suitable liquid for dissolving the effervescent form are, for example, about 1 to about 20 ml, about 5 to 15 ml, and about 10 ml.

Any suitable effervescent solid forms and powders (e.g., one known in the art) can be utilized including the formulations disclosed herein. Effervescent solid forms generally include an effervescent base containing, by way of non-limiting example, at least one alkaline earth metal carbonate and, optionally, an acid. In some instances, when mixed in an aqueous environment, the acid and carbonate react to liberate carbon dioxide gas, thereby causing effervescence. Alternatively, oral administration of the effervescent dosage form may not require an acid because of the acidity of the stomach. Non-limiting examples of alkaline earth metal carbonates include sodium carbonate, sodium bicarbonate, magnesium carbonate, calcium carbonate, potassium carbonate, mixtures thereof, and the like. Suitable acids include, but are not limited, to citric acid and salts (sodium citrate, disodium citrate), tartaric acid and salts, acetic acid and salts, lactic acid and salts, mixtures thereof, and the like.

Additional excipients for effervescent solid forms and powders include optional sweeteners, flavor agents, taste masking agents, preservatives, effervescents, disintegrants, binders, fillers, drug stabilizers, and other known compatible ingredients for effervescent forms. Sweeteners include any sugars (sucrose, high fructose corn syrups), sugar alcohols (mannitol, xylitol, sorbitol), non-saccharide based sweeteners (aspartame, saccharin, sucralose), and combinations thereof. In certain instances, the effervescent solid form or powder contains aspartame. In certain instances, the effervescent solid form or powder contains mannitol. Flavoring agents, include but are not limited to “cool flavoring” agents such as oils of mints, menthols, camphors, N,2,3-trimethyl-2-isopropyl butanamide (WS-23), 2-acetoxy-1,8-cineole, carvyl propionate, and the like; fruit flavoring agents, chocolate flavoring agents and other flavors suitable for effervescent solid forms or powders. In certain instances, the effervescent solid form or powder contains N,2,3-trimethyl-2-isopropyl butanamide.

Drug stabilizers in effervescent solid forms and powders are dependent on the drug in the formulation and include and solubilizers and/or emulsifiers. Suitable solubilizers and emulsifiers for corticosteroids include polyvinylpyrrolidone (K-25), dextrins and cyclodextrins, tyloxapol, gelatin, castor oil derivatives, substituted celluloses, cholesterol, colloidal silicon dioxide, polyoxyethylene sorbitan fatty acid esters, polyethylene glycols, dodecyl trimethyl ammonium bromide, polyvinyl alcohol, sodium lauryl sulfates, poloxamers, poloxamines, charged phospholipids, polyethylene glycol (e.g., Macrogol 6000), docusate sodium, combinations thereof, and the like. In certain instances, the effervescent solid form or powder contains polyvinylpyrrolidone. In certain instances, the effervescent solid form or powder contains docusate sodium. In certain instances, the effervescent solid form or powder contains a polyethylene glycol (e.g., Macrogol 6000).

In certain embodiments, a composition used in a process described herein includes any therapeutic agent described herein formulated in a composition or dosage form of any of U.S. Pat. No. 6,913,779, U.S. Pat. No. 6,596,311, U.S. Pat. No. 6,509,034, U.S. Pat. No. 6,261,602, U.S. Pat. No. 6,139,865, U.S. Pat. No. 5,709,866, U.S. Pat. No. 5,639,475, WO 2009/102830, WO 2009/086046, WO 2009/076361, WO 2009/006516, or WO 2010/009961, each of which is incorporated herein for such disclosure.

### Diseases

In some embodiments, provided herein are methods of treating, preventing, or alleviating disorders or symptoms associated with the gastrointestinal tract, e.g., the esophagus. In certain embodiments, provided herein are methods of treating diseases or conditions of the gastrointestinal tract, e.g., the esophagus, by administering a composition described herein. In specific embodiments, administration of the composition described herein treats, prevents, or alleviates the gastrointestinal disorder (including symptoms of a disease or disorder that present in the gastrointestinal tract). Disorders of the gastrointestinal tract include, by way of non-limiting example, gastrointestinal inflammation, gastrointestinal cancer, gastrointestinal infection (e.g., bacterial or fungal), gastrointestinal motility dysfunction (e.g., esophageal dysmotility), or gastrointestinal lesions, wounds or contusions. More specifically, disorders of the gastrointestinal tract include, by way of non-limiting example, esophageal inflammation, esophageal cancer, esophageal infection (e.g., bacterial or fungal), esophageal motility dysfunction, or esophageal lesions, wounds or contusions. Diseases or conditions of the gastrointestinal tract include, by way of non-limiting example, any chronic inflammatory or malignant state that involves the gastrointestinal tract (e.g., the esophagus, stomach and/or digestive tract) and responds to steroid therapy. The methods of the present invention are useful, for example, for treating, preventing and alleviating the inflammation associated with or symptoms of eosinophilic esophagitis (EoE), intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, eosinophilic gastric outlet obstruction and related inflammation, graft vs. host disease, a skin disease with esophageal involvement, bullous pemphigoid, pemphigus vulgaris, epidermolysis bollosa, Stevens-Johnson syndrome, Behcet's disease, sarcoidosis, idiopathic esophagitis, eosinophilic gastritis, M\acute{ε}n\acute{ε}trier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), inflammatory bowel diseases involving the esophagus, reflux esophagitis, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, acute esophageal inflammation secondary to caustic/irritant ingestion, persistent/recurrent esophageal strictures secondary to caustic/irritant, conditions due to ingestion, systemic diseases, congenital diseases, post-surgery inflammation, and gastro enteritis. The methods of the present invention are also useful, for example, for treating, preventing and alleviating inflammation associated with or symptoms of reflux esophagitis, gastroesophageal reflux disease (GERD), nonerosive reflux disease (NERD), Barrett's Esophagus, and/or erosive esophagitis. The methods of the present invention are also useful, for example, for treating, preventing and alleviating, by way of non-limiting example, celiac disease, drug allergy, connective tissue diseases, graft-vs-host disease, oral chronic graft-versus-host disease, radiation injury, chemical injury, orallesophageal mucositis, oral/esophageal mucositis in cancer patients, and pharyngitis.

In certain embodiments, provided herein is a method of treating gastrointestinal inflammation (e.g., inflammation of the esophagus) in an individual by administering to an individual in need thereof a composition described herein, wherein the therapeutic agent can be a histamine (e.g., H1, H2, and/or H3) receptor ligand, a transient lower esophageal sphincter relaxation (TLESR)-reducing agent, a prokinetic serotonergic agent, a potassium-competitive acid blocker (P-CAB), a mucosal protectant, an anti-gastrin agent, a leukotriene antagonist, a mast cell inhibitor, a mast cell stabilizer, an immunomodulator, a biologic, an anti-asthmatic agent, a non-steroidal anti-inflammatory drug (NSAID), corticosteroid, mGluR₅ antagonists, acetylcholine modulator, 5HT₄ receptor agonist, 5HT₃ receptor antagonist, 5HT₁ receptor antagonist, an antibiotic, an antiseptic, an anesthetic, or a combination thereof. In specific embodiments, gastrointestinal inflammation treated according to the methods described herein include, by way of non-limiting example, eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behcet's disease, sarcoidosis, idiopathic esophagitis, eosinophilic gastritis, M\acute{ε}n\acute{ε}trier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), inflammatory bowel diseases involving the esophagus, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, reflux esophagitis, acute esophageal inflammation secondary to caustic/irritant ingestion, persistent/recurrent esophageal strictures secondary to caustic/irritant, conditions due to ingestion, systemic diseases, congenital diseases, post-surgery inflammation, gastro enteritis, gastroesophageal reflux disease (GERD), nonerosive reflux disease (NERD), Barrett's Esophagus, and/or erosive esophagitis.

In some embodiments, provided herein is a method of treating cancer of the gastrointestinal tract (e.g., esophageal cancer) in an individual by administering to an individual in need thereof a composition described herein, wherein the therapeutic agent is a chemotherapeutic agent.

In certain embodiments, provided herein is a method of treating gastrointestinal (e.g., esophageal) motility dysfunction in an individual by administering to an individual in need thereof a composition described herein, wherein the therapeutic agent is a pro-motility agent, anti-motility agent, or a combination thereof.

In some embodiments, provided herein is a method of treating gastrointestinal (e.g., esophageal) infection in an individual by administering to an individual in need thereof a composition described herein, wherein the therapeutic agent is an antibiotic or antimicrobial agent. In specific embodiments, the antimicrobial agent is an anti-bacterial agent or an anti-fungal agent. In further or alternative embodiments, the infection is a bacterial or fungal infection.

In certain embodiments, provided herein is a method of treating eosinophilic esophagitis (EoE) in an individual by administering to an individual in need thereof a composition described herein, wherein the therapeutic agent is, by way of non-limiting example, a corticosteroid, a leukotriene antagonist, a mast cell stabilizer/inhibitor, an immunomodulator, a biologic, or the like, or combinations thereof.

It will be appreciated that reference herein to treatment extends to prophylaxis as well as the treatment of inflammation or other disorders.

In certain embodiments, provided herein is a method of treating, preventing or alleviating disorders of the gastrointestinal tract, including, by way of non-limiting example, the esophagus, stomach and/or digestive tract, in an individual comprising orally administering to said individual any of the compositions described herein. In certain embodiments, the oral dosage form comprises a liquid vehicle and is formulated as, e.g., a slurry, suspension, syrup, dispersion, solution, or the like.

In some embodiments, the inflammation treated by the methods and compositions described herein is associated with mast cell inflammation, eosinophilic inflammation and/or neutrophilic inflammation. In some embodiments, individuals (e.g., patients) to be treated with compositions described herein include those that have been diagnosed with eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), an inflammatory bowel disease involving the esophagus, reflux esophagitis, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation, or gastro enteritis. In one non-limiting example, the patient has eosinophilic esophagitis. In some embodiments, individuals (e.g., patients) to be treated with the compositions described herein include those that have been diagnosed with gastroesophageal reflux disease (GERD), nonerosive reflux disease (NERD), Barrett's Esophagus, and/or erosive esophagitis. In some embodiments, individuals to be treated with the compositions described herein suffer from, by way of non-limiting example, gastrointestinal inflammation, gastrointestinal cancer, gastrointestinal infection (e.g., bacterial or fungal), gastrointestinal motility dysfunction, gastrointestinal lesions, wounds or contusions, celiac disease, drug allergy, connective tissue diseases, graft-vs-host disease, oral chronic graft-versus-host disease, radiation injury, chemical injury, oral/esophageal mucositis, oral/esophageal mucositis in cancer patients, and pharyngitis. In some embodiments, the patient is an adult. In other embodiments, the patient is a child or infant. In various aspects, a patient is a child or infant less than 16 years old, less than 12 years old, less than 8 years old, less than 6 years old, less than 4 years old or less than 2 years old.

In some embodiments, provided herein are methods and compositions for treating, preventing and alleviating a disorder that involves esophageal remodeling. Esophageal remodeling can be associated with various types of esophagitis, including but not limited to, eosinophilic esophagitis. In certain instances, remodeling is lamina propria remodeling. In various instances, remodeling includes one or more properties of vascular activation: expression of VCAM-1; level of interstitial edema and TGFβ₁ activation. In some embodiments, provided herein are methods and compositions for reducing esophageal remodeling. In certain instances reduced esophageal remodeling is accompanied by decreased fibrosis, TGFβ1 and pSmad2/3 positive cells, and decreased vascular activation, or a combination thereof. In some embodiments, provided herein are methods and compositions for reducing esophageal remodeling and/or reducing epithelia eosinophils.

In some embodiments, initial treatment continues, for example, for about 3 days to 2 weeks for an acute condition, or about 4 weeks to about 16 weeks for a chronic condition, or about 8 weeks to about 12 weeks for a chronic condition. In various embodiments, longer therapy is needed, such as, for example, therapy similar to chronic therapy for persistent asthma. In some aspects of the present invention, patients are, for example, be treated for up to 6 months, or up to one year. In certain aspects, maintenance treatments last up to or longer than one year. In some embodiments, patients are treated on a maintenance basis or on an as needed basis during a problematic episode, depending on the severity of the condition. In certain embodiments, patients are treated on a rotating treatment basis, where treatment is provided for a period of time and then the patient is taken off of the drug for a period before treatment resumes again. When off the drug, the patient may be given no treatment, treatment with another medication, dietary therapy, or treatment with a reduced dosage. In certain embodiments, patients are given treatment with a higher dose of the composition until a desired reduced disease state is achieved, and then continued on a lower dose of the composition. In certain embodiments, a patient combines treatment with a composition described herein with a treatment with another medication, and/or dietary therapy. In certain embodiments, patients are given treatment with a higher dose of the composition until a desired reduced disease state is achieved, and then continued on a lower dose of the composition.

In some embodiments, provided herein is a process of diagnosing an individual with gastrointestinal a gastrointestinal disorder by (i) detecting and/or measuring symptoms of the disorder in an individual prior to administering to the individual a composition described herein; (ii) administering to the individual a composition described herein; (iii) detecting and/or measuring symptoms of the individual following administration of the composition; and (iv) comparing the symptoms measured or detected prior to and following administration of a composition described herein. If the symptoms exhibited by the individual are reduced (e.g., by a statistically significant or clinically relevant amount), a positive diagnosis occurs. In specific embodiments, the process of diagnosing an individual with gastrointestinal inflammation is diagnosing an individual with eosinophilic esophagitis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of preventing or alleviating (i) gastrointestinal inflammation, (ii) gastrointestinal tissue remodeling, and/or (iii) symptoms associated therewith in an individual comprising orally administering to said individual a composition comprising at least one therapeutic agent and at least one excipient that increases the interaction of the composition with a gastrointestinal surface, wherein the composition is in the form of a powder, granules, micropellets, nanopellets, microparticles, nanoparticles, a tablet, an effervescent tablet, a melting tablet, a disintegrating tablet, a dissolving wafer, an orally disintegrating tablet, a foam, a gel, a gum, a wafer, a dissolving wafer, a disintegrating wafer, a melting wafer, a lozenge, a film, a patch, a solid solution, an emulsion, a liquid or semi-liquid solution, or a combination thereof.

2. The method of claim 1, wherein the tablet is a compressed tablet, a multiple compressed tablet, an oral disintegrating tablet, a sugar coated tablet, a chocolate-colored tablet, a film coated tablet, an enteric coated tablet, a fast dissolving tablet, a chewable tablet, a buccal tablet, a sublingual tablet, or combinations thereof.

3. The method of claim 1, wherein the gastrointestinal inflammation is esophageal inflammation and the gastrointestinal surface is esophageal epithelium.

4. The method of claim 1, wherein the gastrointestinal tissue remodeling is esophageal remodeling of the lamina propria.

5. The method of claim 1, wherein the at least one therapeutic agent comprises a corticosteroid.

6. The method of claim 5, wherein the corticosteroid is selected from the group consisting of aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol, or pharmaceutically acceptable salts or esters thereof, or combinations thereof.

7. The method of claim 5, wherein the corticosteroid is budesonide.

8. The method of claim 1, wherein the at least one therapeutic agent is selected from the group consisting of a histamine receptor ligand including a histamine antagonist, a transient lower esophageal sphincter relaxation (TLESR)-reducing agent, a promotility agent, a prokinetic serotonergic agent, a potassium-competitive acid blocker (P-CAB), a mucosal protectant, an anti-gastrin agent, an anti-inflammatory agent, an antibacterial agent including an antibiotic, an antifungal agent, a wound healing agent, a chemotherapeutic agent, a monoclonal antibody, an antiemetic agent, erythropoietin, a synthetic erythropoietin, a leukotriene antagonist, a mast cell inhibitor, a mast cell stabilizer, an immunomodulator, an anti-asthmatic agent, a non-steroidal anti-inflammatory drug (NSAID), a corticosteroid, a mGluR₅ antagonist, an acetylcholine modulator, a 5HT₄ receptor agonist, a 5HT₃ receptor antagonist, a 5HT₁ receptor antagonist, an antiseptic agent, an anesthetic, or combinations thereof.

9. The method of claim 1, wherein the at least one therapeutic agent comprises aminosalicyclic acids.

10. The method of claim 1, wherein the at least one therapeutic agent comprises a corticosteroid and an acid inhibitor.

11. The method of claim 1, wherein the at least excipient that increases the interaction of the composition with a gastrointestinal surface comprises a viscosity-increasing excipient.

12. The method of claim 1, wherein the composition is in the form of powders or granules, wherein the at least one therapeutic agent and the at least one excipient are mixed and sprayed onto a powdered or granular pharmaceutical support material selected from the group consisting of microcrystalline cellulose and mixtures of microcrystalline cellulose with lactose.

13. The method of claim 1, wherein the composition is in the form of micropellets, wherein the composition comprising:
(a) from about 10 to 95% by weight of the at least one therapeutic agent;
(b) from about 5 to about 90% by weight of at least one pharmaceutically acceptable binding agent;
(c) from about 0 to about 10% by weight of at least one pharmaceutically acceptable excipient; and
(d) one or more enzyme-friendly organic solvents in an amount sufficient to form an extrudable mixture.

14. The method of claim 1, wherein the composition comprises microparticles, wherein the composition comprising:
(a) microparticles comprising the at least one therapeutic agent;
(b) a film coated composed of
(i) a pH-independent water-insoluble polymer accounting for 60% or more but less than 80% of the film and
(ii) a pH-independent water-soluble substance accounting for more than 20% to 40% or less of the film,
wherein the average particle diameter is 350 μm or less.

15. The method of claim 2, wherein the composition comprises an oral disintegrating tablet, wherein the composition further comprising:
(a) a methacrylic polymer; and
(b) one or more additional excipients;
wherein said methacrylic polymer and the one or more additional excipient form a single coating layer over the at least one therapeutic agent; and
wherein the one or more additional excipient is present as an extragranular ingredient.

16. The method of claim 2, wherein the composition comprises an oral disintegrating tablet, wherein the composition further comprising:
(a) an effervescent base, wherein said effervescent base and comprises
(i) at least one alkaline earth metal carbonate,
(ii) an organic edible acid, and
(iii) an alkali metal salt of citric acid; and optionally, and
(b) a pharmaceutically acceptable auxiliary ingredient.

17. The method of claim 2, wherein the composition comprises an oral disintegrating tablet, wherein the composition comprising:
(a) 5-40 weight percent of the at least one therapeutic agent;
(b) 40-90 weight percent of a filler;
(c) 0.5-10 weight percent of a binder;
(d) 0.5 to 10 weight percent of a flavoring agent; and
(e) 1-15 weight percent of a disintegrant.

18. The method of claim 2, wherein the composition comprises a chewable tablet, wherein the composition comprising:
(a) a therapeutically-effective amount of the at least one therapeutic agent dispersed in a solid pharmaceutically-acceptable lipid coating, which lipid is solid at ambient temperature, or mixtures of said lipids, wherein the lipid is present in the composition in an amount of from 5-50 percent by weight.
(b) a matrix for said drug and lipid, said matrix consisting essentially of:
(i) one or more granulating agents,
(ii) a rapid dispersal agent in an amount of from about 2 to about 20 weight percent of the composition, wherein the rapid dispersal agent is blended with the solidified lipid coated drug, and
(iii) optionally minor amounts of additives selected from the group consisting of
flavoring agents, coloring agents, buffering agents, sweeteners, oils, and surfactants.
The

19. A method of preventing or alleviating (i) gastrointestinal inflammation, (ii) gastrointestinal tissue remodeling, and/or (iii) symptoms associated therewith in an individual comprising:
(a) combining a solid composition with a pharmaceutically acceptable liquid to prepare a pharmaceutically administrable composition, the solid composition comprising at least one therapeutic agent and at least one excipient that increases the interaction of the administrable composition with a gastrointestinal surface; and
(b) orally administering to said individual the administrable composition.

20. The method of claim 19, wherein the solid composition is in the form of a powder, granules, micropellets, nanopellets, microparticles, nanoparticles, a tablet, an effervescent tablet, a disintegrating tablet, sachet, or a combination thereof.

21. The method of claim 19, wherein the pharmaceutically acceptable liquid comprise water, alcohol, or a combination thereof.

22. The method of claim 19, wherein the gastrointestinal inflammation is esophageal inflammation and the gastrointestinal surface is esophageal epithelium and/or the gastrointestinal tissue remodeling is esophageal remodeling of the lamina propria.

23. The method of claim 19, wherein the at least one therapeutic agent comprises a corticosteroid.

24. The method of claim 23, wherein the corticosteroid is selected from the group consisting of aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol, or pharmaceutically acceptable salts or esters thereof, and combinations thereof.

25. The method of claim 19, wherein the at least one therapeutic agent is selected from the group consisting of a histamine receptor ligand including a corticosteroid, a histamine antagonist, a transient lower esophageal sphincter relaxation (TLESR)-reducing agent, a promotility agent, a prokinetic serotonergic agent, a potassium-competitive acid blocker (P-CAB), a mucosal protectant, an anti-gastrin agent, an anti-inflammatory agent, an antibacterial agent including an antibiotic, an antifungal agent, a wound healing agent, a chemotherapeutic agent, a monoclonal antibody, an antiemetic agent, erythropoietin, a synthetic erythropoietin, a leukotriene antagonist, a mast cell inhibitor, a mast cell stabilizer, an immunomodulator, an anti-asthmatic agent, a non-steroidal anti-inflammatory drug (NSAID), a corticosteroid, a mGluR₅ antagonist, an acetylcholine modulator, a 5HT₄ receptor agonist, a 5HT₃ receptor antagonist, a 5HT₁ receptor antagonist, an antiseptic agent, an anesthetic, and combinations thereof.
